(19) United States
(12) Patent Application Publication
(10) Pub. No.: US 2024/0024466 A1
(43) Pub. Date: Jan. 25, 2024

(54) LIPIDOID COMPOSITIONS AND METHODS OF USE THEREOF

(71) Applicant: Trustees of Tufts College, Medford, MA (US)

(72) Inventors: Qiaobing Xu, Medford, MA (US); Jinjin chen, Medford, MA (US)

(21) Appl. No.: 18/265,561

(22) PCT Filed: Dec. 7, 2021
(86) PCT No.: PCT/US21/62273

§ 371 Date: Jun. 6, 2023

Related U.S. Application Data
(60) Provisional application No. 63/122,229, filed on Dec. 7, 2020.

(51) Int. Cl. A61K 39/39; A61K 39/215; A61P 37/04

(52) U.S. Cl. CPC A61K 39/39; A61K 39/215; A61P 37/04; A61K 2039/55555

(57) ABSTRACT

Disclosed are lipidoid compositions that are capable of treating or preventing certain diseases (e.g., cancer or viral infections). Also disclosed are pharmaceutical compositions, comprising the lipidoid compositions. The present disclose also relates to methods of using the lipidoid compositions, and related kits comprising the lipidoid compositions.

### RELATED APPLICATIONS

This application claims the benefit of priority to U.S. Provisional Application No. 63/122,229, filed Dec. 7, 2020; the contents of which are fully incorporated by reference herein.

### GOVERNMENT SUPPORT

This invention was made with Government support under Grant No. R01 EB027170-01 awarded by the National Institutes of Health (NIH). The Government has certain rights in the invention.

### BACKGROUND

Cancer vaccines have been in development for several decades, providing new options for the treatment of cancers. Though this approach shows considerable promise for the eradication of cancer, only one cell-based vaccine has been approved by the US Food and Drug Administration (FDA) to date, and the overall rate of clinical benefit is still low. Two major obstacles have hindered the development of cancer vaccines: the high variability of tumor-associated antigens (TAAs) between different tumors, and even different patients with the same tumors, and immunosuppressive tumor microenvironments. Though personalized vaccines based on neoantigens and immune checkpoint inhibitor have been developed to overcome these challenges, these procedures were complex, costly, and only effective in small populations. In situ vaccination is considered a promising alternative strategy of cancer vaccination without the need to identify and isolate the TAAs. By local administration of therapeutic or immunomodulatory agents, in situ vaccination aims to harness abundant TAAs and activate the immunosuppressive tumor microenvironments, thus offering the possibility of wide application.

Use of oncolytic virus is the most common method for in situ vaccination, and have been explored in clinical trials against metastatic melanoma. However, the excessive activation of immune system by oncolytic virus has become the major concern about the application in humans, which might lead to severe side effects, such as cytokine release syndrome (CRS). To date, some other in situ vaccination strategies, including photothermal therapy (PTT), radiation therapy (RT), agonist immunotherapy, and even chemotherapy, were also shown to induce effective immune response. However, PTT, RT, and chemotherapy were only efficient in production of tumor antigens by induction of immunogenetic cell death, but the delivery and presentation of these released antigens was still limited by immune tolerance of tumor microenvironments. The in situ application of immune agonists can boost the immune response but is deficient in production of tumor antigens. In order to generate an effective anti-tumor immune response, an in situ vaccine should ideally be able to induce immunogenic cancer cell death, facilitate the release of TAAs, enhance antigen presentation, and promote the activation of anti-tumor T cell responses.

In view of the foregoing, there is an ongoing, unmet need for new vaccines.

### SUMMARY

In situ vaccination is a promising strategy for cancer immunotherapy owing to its convenience and the ability to induce numerous tumor antigens. However, the advancement of in situ vaccination techniques has been hindered by low cross-presentation of tumor antigens and the immunosuppressive tumor microenvironment. To balance the safety and efficacy of in situ vaccination, a lipidoid composition, e.g., nanoparticles (LNP) were designed to achieve simultaneously enhancing cross-presentation and STING activation. From screening the library of the LNPs, 93-O17S-F was identified. 93-O17S-F promotes both the cross-presentation of tumor antigens and the intracellular delivery of cGAMP (STING agonist). Intratumor injection of 93-O17S-F/cGAMP in combination with pre-treatment of doxorubicin exhibited excellent antitumor efficacy with 35% of mice exhibiting total recovery from a primary B16F10 tumor and 710% of mice with a complete recovery from a subsequent challenge, indicating the induction of an immune memory against the tumor. Thus, the disclosure herein provides a promising strategy for in situ cancer immunotherapy.

In one aspect, the present disclosure provides lipidoid compositions (e.g., nanoparticles) nanoparticles comprising a plurality of lipidoids; and an adjuvant (e.g., an immune modulator), an antigen, or a nucleic acid.

In another aspect, the present disclosure provides pharmaceutical compositions comprising the lipidoid compositions disclosed herein.

In another aspect, the present disclosure provides methods of treating or preventing a disease or disorder in a subject in need thereof with the lipidoid compositions disclosed herein.

In another aspect, the present disclosure provides methods of treating cancer in a subject in need thereof with the lipidoid compositions disclosed herein.

In another aspect, the present disclosure provides methods of treating or preventing a viral infection in a subject in need thereof with the lipidoid compositions disclosed herein.

In another aspect, the present disclosure provides kits comprising the lipidoid compositions disclosed herein.

### BRIEF DESCRIPTION OF THE DRAWINGS

FIG. 1 shows the mechanism of LNP system-mediated cross-presentation and STING activation. (I) Low dose of DOX induced immunogenic cancer cell death. (II) The tumor antigens were released to after the administration of low dose of DOX. (III) The released antigens were captured by LNP/cGAMP. (IV) The tumor antigens and cGAMP were delivered into APC cells via endocytosis. (V) The tumor antigens and cGAMP were escaped from endo/lysosome for further cross-presentation and STING activation.

FIGS. 2A-2G show that the adjuvant effect and enhanced cross-presentation of LNP. FIG. 2A shows the route for the screening of LNP library by prime-boost route. FIGS. 2B-2D show OVA-specific IgG, IgG1, and IgG2c antibody titers after immunization with OVA-loaded LNPs. n=3. FIG. 2E shows that the enhanced cytoplasmic delivery of antigens by LNPs upregulated the cross-presentation. FIG. 2F shows typical flowcytometry data of the expression of SIINFEKL-MHC I complex on DC2.4 cells after incubation of different formulation of OVA. FIG. 2G shows the mean fluorescence intensity (MFI) of labelled SIINFEKL-MHC I complex calculated by flowcytometry. n=4.

FIGS. 3A-3E show the enhanced STING activation by cytoplasm delivery of cGAMP in vitro. FIG. 3A shows the activation of STING pathway by cytoplasmic delivery of cGAMP using 93-O17S-F. FIG. 3B show the subcellular distribution of cGAMP^{Fluo} and lysosome in RAW264.7 and DC2.4 cells after incubation of free cGAMP^{Fluo} or 93-O17S-F/cGAMP^{Fluo} for 4 h. FIGS. 3C & 3D show the relative expressions of ifnb1 and cxcl10 genes in RAW264.7 and DC2.4 cells after incubation of 93-O17S-F/cGAMP for 4 h. FIG. 3E shows the concentration of IFN-β in the medium of DC2.4 cells after incubation of 93-O17S/cGAMP for 4 and 24 h.

FIGS. 4A-4I show that LNP enhanced STING activation and shifted immunocellular composition of the tumor microenvironment in vivo. FIG. 4A shows the capture of the tumor antigens by 93-O17S-F. FIG. 4B shows the diameters and zeta potentials of 93-O17S-F and tumor lysate complex at different weight ratio. FIG. 4C shows the enhanced delivery of OVA^{Alexa647} to draining lymph nodes after being captured by 93-O17S-F in vivo. FIG. 4D shows the route of the in vivo STING activation experiments. FIG. 4E & 4F show the relative expression of ifnb1 and cxcl10 genes in B16F10 tumors after the administration of 93-O17S-F/cGAMP for 6 hours. n=6, *P≤0.05. FIG. 4G shows the activation of STING pathway recruited the immune cells to tumor sites. FIG. 4H shows the cell numbers of CD4⁺ and CD8⁺ T cells at tumor sites after the administration of 93-O17S-F/cGAMP for 48 h. n=5, *P≤0.05, ***P≤0.01. FIG. 4I shows the cell numbers of DCs and macrophages at tumor sites after the administration of 93-O17S-F/cGAMP for 48 h. n=5, *P≤0.05.

FIGS. 5A-5G show the anti-tumor therapeutic effect of 93-O17S-F/cGAMP. FIG. 5A shows the route of in situ vaccination by 93-O17S/cGAMP. FIG. 5B shows photographs of B16F10 xenografted tumors at day 6. FIG. 5C shows the tumor volumes of B16F10 xenografted tumors after the treatment of different formulations. n=6, ***P<0.001. FIG. 5D shows the individual tumor volumes after the treatment of different formulations. FIG. 5E shows the survival rates of mice bearing B16F10 xenografted tumors. FIG. 5F shows the route of tumor rechallenge assay. FIG. 5G shows the percentage of total recovery of primary and rechallenged tumor inoculated mice.

FIG. 6 shows two synthesis routes based on acrylate and epoxyethyl groups.

FIG. 7A show the amine heads and tails of the lipidoids used in the library screening.

FIG. 7B shows the IgG1 titer in the blood of the mice vaccinated by different LNP/OVA complexes (n=2).

FIG. 8 shows typical OD values of the ELISA assay for determination of antibody titers.

FIG. 9A shows the detailed sequences of the primers of gapdh, cxcl10, and ifnb1 genes.

FIG. 9B shows semiquantitative PCR of target genes from DC2.4 cell mRNA.

FIG. 10 shows TEM images of 93-O17S F and 93-O17S F/tumor lysate complexes.

FIG. 11 shows gating information for the evaluation of cell composition of B16F10 tumors after in situ vaccination.

FIG. 12 shows the design of mRNA for vaccine design.

### DETAILED DESCRIPTION

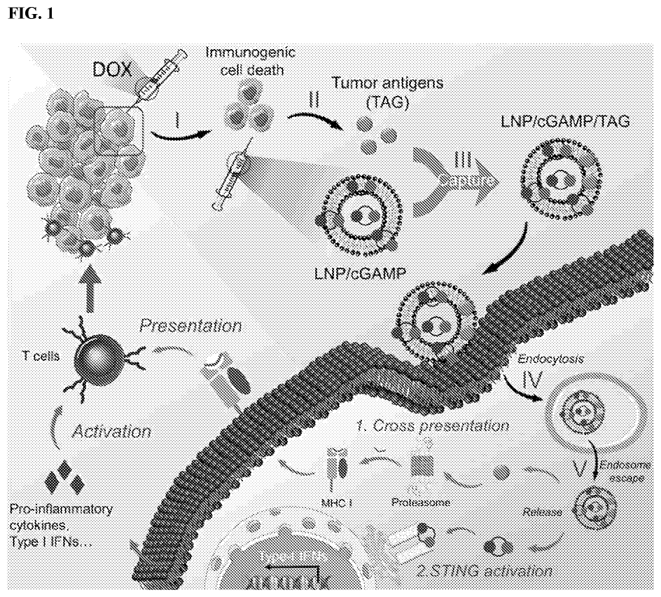

In situ vaccination by lipidoid nanosystem with enhanced cross-presentation and STING-activation increases therapeutic efficacy for solid tumors. Herein, we designed a lipidoid-based nanosystem that can enhance the effect of in situ vaccination by promoting cross-presentation of TAAs and activation of interferon genes (STING) pathway. As shown in FIG. 1, the primary tumors are injected with a small dose of doxorubicin (DOX), which induces tumor immunogenetic death and the release of TAAs.¹⁹ Then the 2′5′-3′5′ cyclic guanosine monophosphate-adenosine monophosphate (cGAMP)-loaded lipidoid composition (e.g., a nanoparticle) (LNP/cGAMP) is injected to the apoptotic site of tumor to capture the released tumor antigens via electrostatic interaction. The LNP encapsulated with both tumor antigens and cGAMP are delivered to the antigen presentation cells (APCs) through enhanced endocytosis. More importantly, the antigens and cGAMP could be released into cytoplasm of the APCs via endo/lysosome escape effect of LNP. The released tumor antigens are then degraded by ubiquitin-proteasome system and presented by the major histocompatibility complex (MHC) class I to activate T cells.²⁰ The released cGAMP in the cytoplasm could activate the STING pathway and production of type I interferon (IFN) and other pro-inflammatory cytokines, which also promote activation of T cells.²¹ The integration of enhanced crossing-presentation and STING activation can promote the in situ vaccination for tumor immunotherapy. Comparing with oncolytic virus-based cancer immunotherapy, the synthetic lipidoid compositions have considerable advantages, including the better safety profile, and easier for massive production. Comparing with other non-viral based in situ vaccination systems, the present system has the advantages in antigen capturing, delivery and cross-presentation, hence provides better therapeutic effects.

In one aspect, the present disclosure provides lipidoid compositions comprising a plurality of lipidoids; and an adjuvant, an antigen, or a nucleic acid.

In certain embodiments, each lipidoid independently comprises an amine head and a hydrophobic tail.

In certain embodiments, each amine head independently comprises an alkylamine, an arylamine, a heteroarylamine, or a heterocyclylamine. In certain embodiments, each amine head independently comprises a C₄-C₂₀alkyl chain comprising 1, 2, 3, 4, 5, 6, 7, or 8 nitrogen containing moieties. In certain embodiments, each amine head independently comprises a C₄-C₂₀alkyl chain comprising 1, 2, 3, or 4 nitrogen containing moieties.

In certain embodiments, the nitrogen containing moieties are primary amines (e.g., NH2), alkyl amines (e.g., mono- or di-alkylamines, such as mono- or dimethyl or mono- or diethylamine), heteroaryl groups (e.g., imidazole or pyridine), or heterocyclyl groups (e.g., piperidine or piperazine).

In certain embodiments, the each amine head is independently substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide. In certain embodiments, each amine head is independently substituted with hydroxyl.

In certain embodiments, each amine head independently comprises

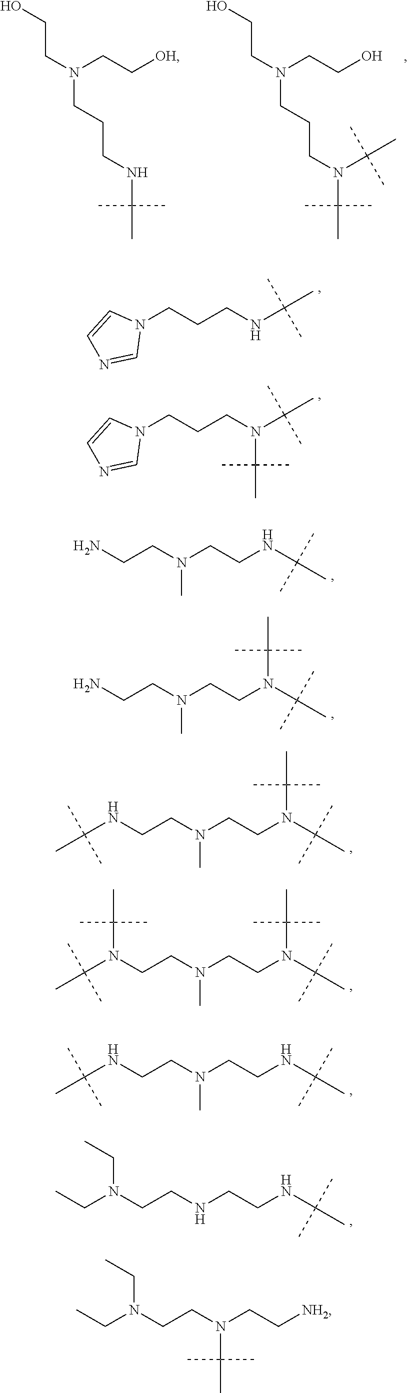

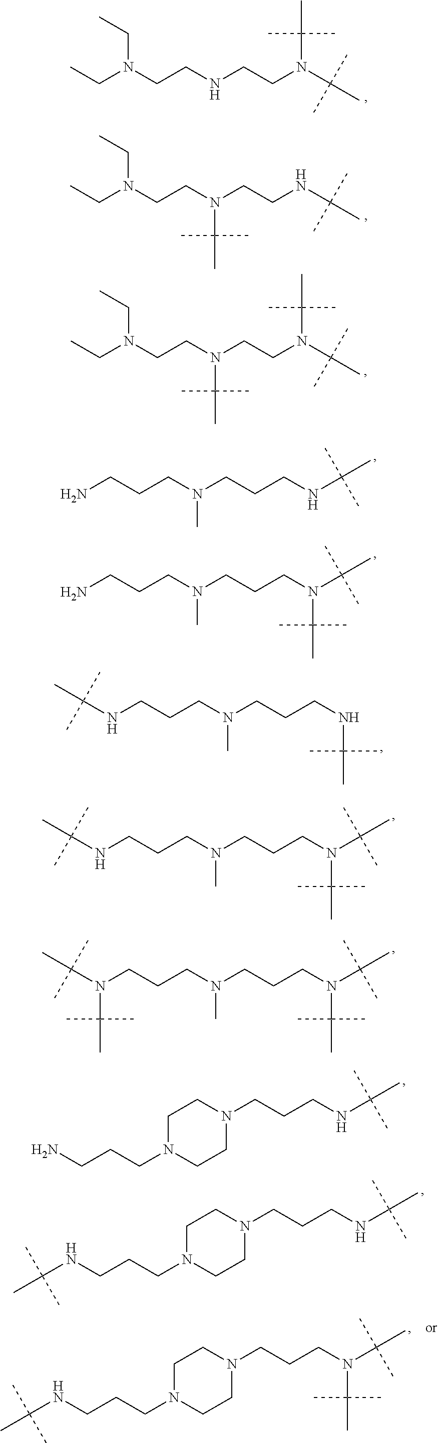

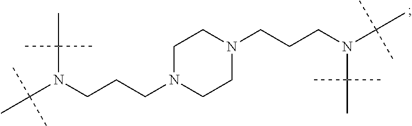

or a pharmaceutically acceptable salt thereof, and
each dashed line represents a connection (e.g., a bond) to the hydrophobic tail.

In certain embodiments, wherein each amine head independently comprises

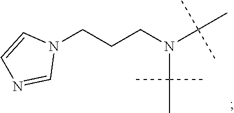

or a pharmaceutically acceptable salt thereof; and
each dashed line represents a connection (e.g., a bond) to the hydrophobic tail.

In certain embodiments, each amine head independently comprises

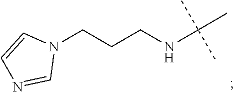

or a pharmaceutically acceptable salt thereof; and
the dashed line represents a connection (e.g., a bond) to the hydrophobic tail.

In certain embodiments, each amine head independently consists essentially of

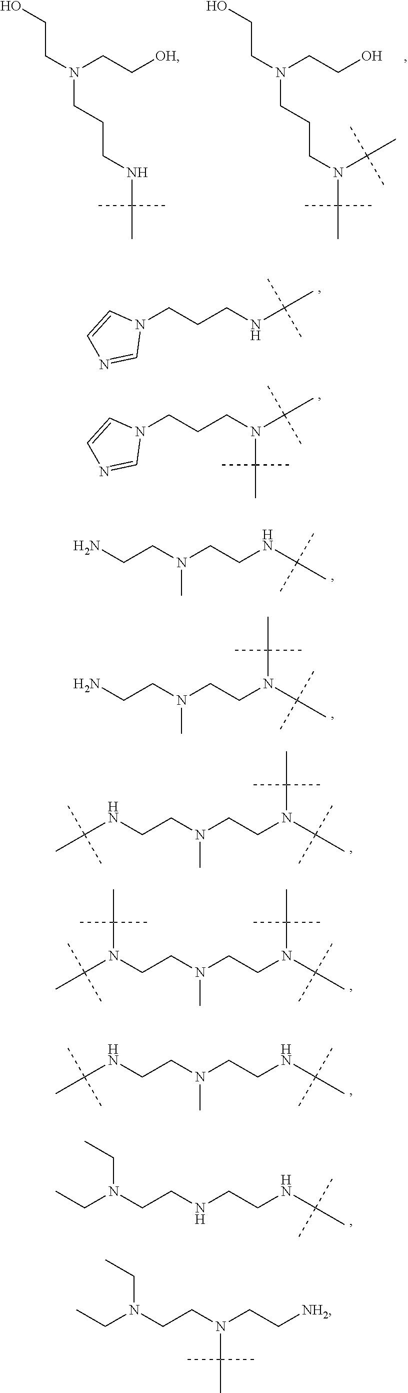

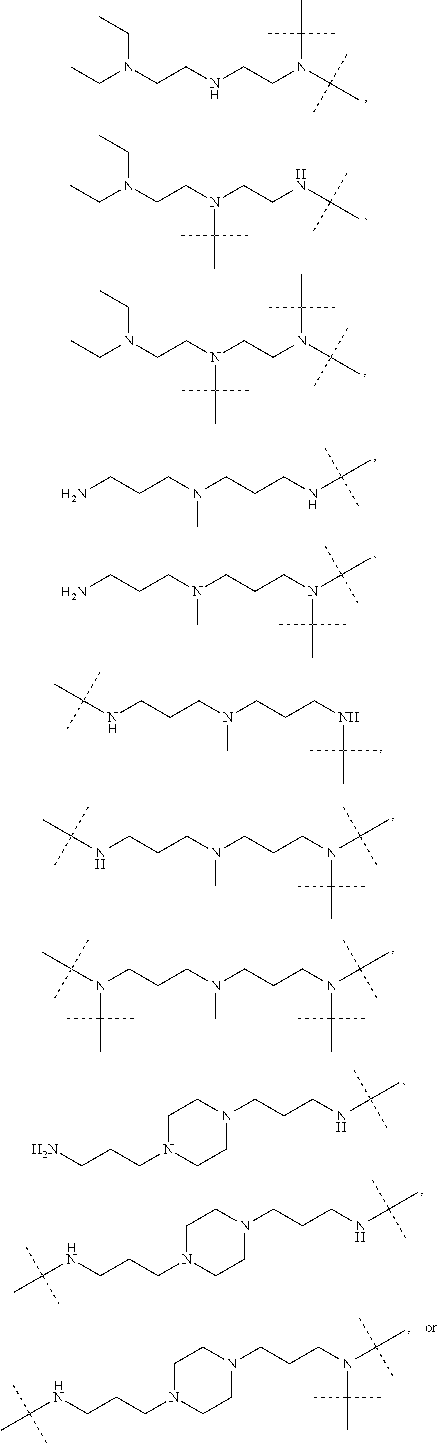

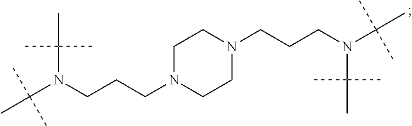

or a pharmaceutically acceptable salt thereof, and
each dashed line represents a connection (e.g., a bond) to the hydrophobic tail.

In certain embodiments, each amine head independently consists essentially of

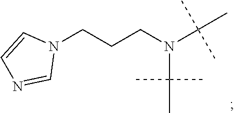

or a pharmaceutically acceptable salt thereof; and
each dashed line represents a connection to the hydrophobic tail.

In certain embodiments, each amine head independently consists essentially of

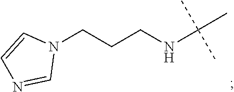

or a pharmaceutically acceptable salt thereof, and
the dashed line represents a connection (e.g., a bond) to the hydrophobic tail.

In certain embodiments, each hydrophobic tail independently comprises a C₁-C₃₀ alkyl chain, a C₁-C₃₀ alkylacyl (e.g., C₄-C₃₀ alkylacyl, C₆-C₃₀ alkylacyl, C₄-C₂₅ alkylacyl, C₆-C₂₅ alkylacyl, C₄-C₂₀ alkylacyl, C₆-C₂₀ alkylacyl, C₄-C₁₈ alkylacyl, or C₆-C₁₈ alkylacyl) chain, a C₁-C₃₀ alkylester (e.g., C₄-C₃₀ alkylester, C₆-C₃₀ alkylester, C₄-C₂₅ alkylester, C₆-C₂₅ alkylester, C₄-C₂₀ alkylester, C₆-C₂₀ alkylester, C₄-C₁₈ alkylester, or C₆-C₁₈ alkylester) chain, or a C₁-C₃₀ alkylamide (e.g., C₄-C₃₀ alkylamide, C₆-C₃₀ alkylamide, C₄-C₂₅ alkylamide, C₆-C₂₅ alkylamide, C₄-C₂₀ alkylamide, C₆-C₂₀ alkylamide, C₄-C₁₈ alkylamide, or C₆-C₁₈ alkylamide) chain. In certain embodiments, each hydrophobic tail independently comprises a C₁-C₃₀ alkyl (e.g., C₄-C₃₀ alkyl, C₆-C₃₀ alkyl, C₄-C₂₅ alkyl, C₆-C₂₅ alkyl, C₄-C₂₀ alkyl, C₆-C₂₀ alkyl, C₄-C₁₈ alkyl, or C₆-C₁₈ alkyl) chain, a C₁-C₃₀ alkylester (e.g., C₄-C₃₀ alkylester, C₆-C₃₀ alkylester, C₄-C₂₅ alkylester, C₆-C₂₅ alkylester, C₄-C₂₀ alkylester, C₆-C₂₀ alkylester, C₄-C₁₈ alkylester, or C₆-C₁₈ alkylester) chain, or a C₁-C₃₀ alkylamide (e.g., C₄-C₃₀ alkylamide, C₆-C₃₀ alkylamide, C₄-C₂₅ alkylamide, C₆-C₂₅ alkylamide, C₄-C₂₀ alkylamide, C₆-C₂₀ alkylamide, C₄-C₁₈ alkylamide, or C₆-Cis alkylamide) chain.

In certain embodiments, at least one carbon atom of the C₁-C₃₀ alkyl (e.g., C₄-C₃₀ alkyl, C₆-C₃₀ alkyl, C₄-C₂₅ alkyl, C₆-C₂₅ alkyl, C₄-C₂₀ alkyl, C₆-C₂₀ alkyl, C₄-C₁₈ alkyl, or C₆-C₁₈ alkyl) is replaced with a heteroatom. In certain embodiments, 1, 2, 3, 4, 5, 6, 7, or 8 carbon atom(s) of the C₁-C₃₀ alkyl (e.g., C₄-C₃₀ alkyl, C₆-C₃₀ alkyl, C₄-C₂₅ alkyl, C₆-C₂₅ alkyl, C₄-C₂₀ alkyl, C₆-C₂₀ alkyl, C₄-C₁₈ alkyl, or C₆-C₁₈ alkyl) is replaced with a heteroatom. In certain embodiments, 1 carbon atom of the C₁-C₃₀ alkyl (e.g., C₄-C₃₀ alkyl, C₆-C₃₀ alkyl, C₄-C₂₅ alkyl, C₆-C₂₅ alkyl, C₄-C₂₀ alkyl, C₆-C₂₀ alkyl, C₄-C₁₈ alkyl, or C₆-C₁₈ alkyl) is replaced with a heteroatom. In certain embodiments, 2 carbon atoms of the C₁-C₃₀ alkyl (e.g., C₄-C₃₀ alkyl, C₆-C₃₀ alkyl, C₄-C₂₅ alkyl, C₆-C₂₅ alkyl, C₄-C₂₀ alkyl, C₆-C₂₀ alkyl, C₄-C₁₈ alkyl, or C₆-C₁₈ alkyl) is replaced with a heteroatom.

In certain embodiments, each heteroatom is independently selected from the group consisting of N (e.g., NH), 0, S, and Se. In certain embodiments, each heteroatom is independently selected from the group consisting of O, S, and Se. In certain embodiments, each heteroatom is independently selected from the group consisting of O, S, and Se. In certain embodiments, each heteroatom is O. In certain embodiments, each heteroatom is S. In certain embodiments, each heteroatom is Se.

In certain embodiments, each hydrophobic tail is independently substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide. In certain embodiments, each hydrophobic tail is independently substituted with halo (e.g., fluoro).

In certain embodiments, each hydrophobic tail independently comprises the formula of

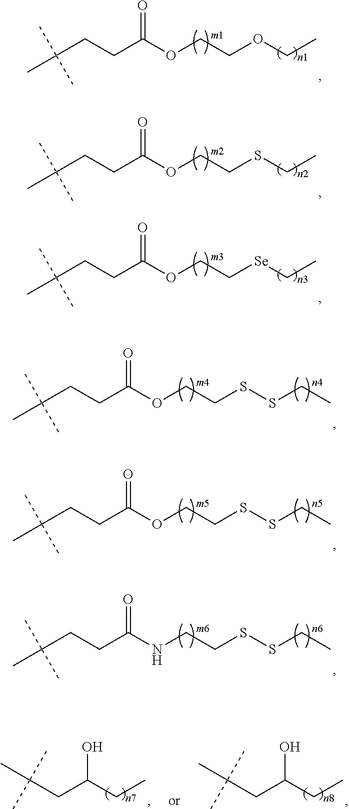

wherein the dashed line represents a connection (e.g., a bond) to the amine head, and wherein:
m1 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m2 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m3 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m4 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m5 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m6 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n1 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n2 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n3 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n4 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n5 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n6 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n7 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15); and
n8 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15).

In certain embodiments, each hydrophobic tail independently comprises

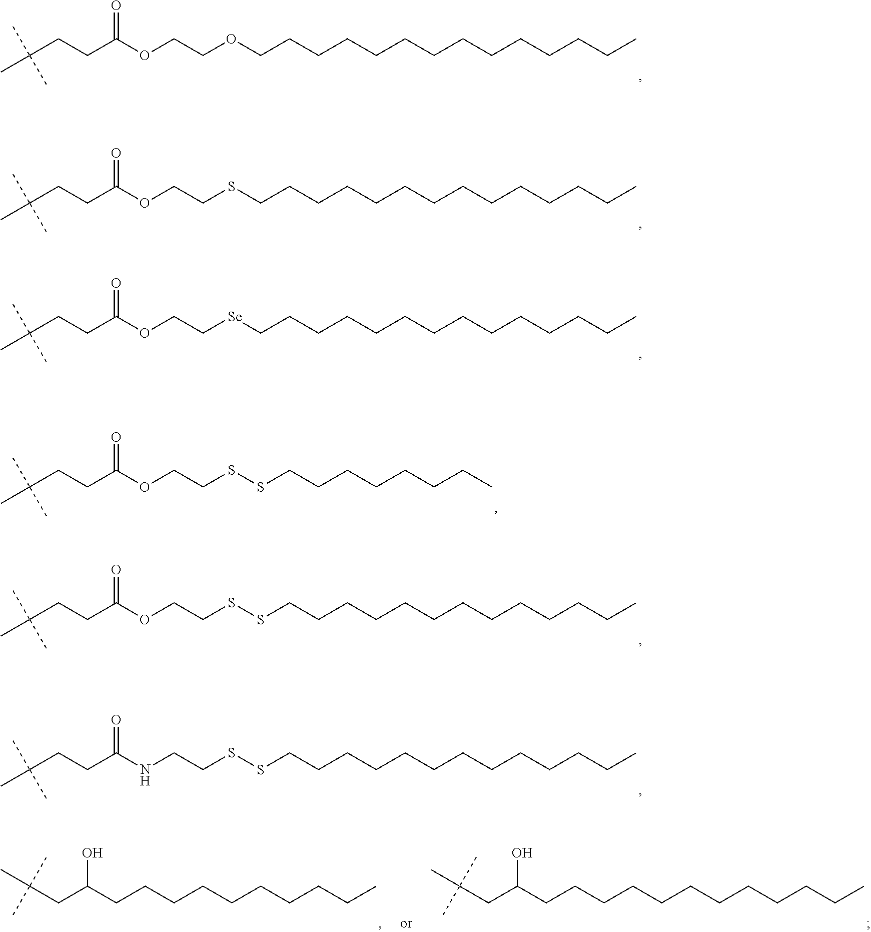

and
the dashed line represents a connection (e.g., a bond) to the amine head.

In certain embodiments, each hydrophobic tail comprises

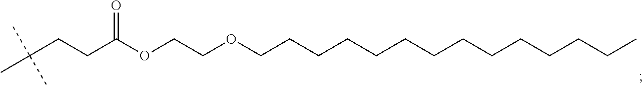

and
the dashed line represents a connection (e.g., a bond) to the amine head.

In certain embodiments, the alkyl chain is substituted with halo (e.g., fluoro).

In certain embodiments, each hydrophobic tail comprises

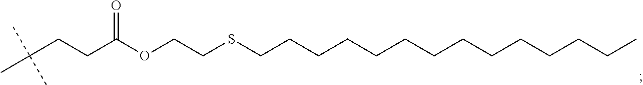

and
the dashed line represents a connection (e.g., a bond) to the amine head.

In certain embodiments, the alkyl chain is substituted with halo (e.g., fluoro).

In certain embodiments, each hydrophobic tail independently consists essentially of the formula of

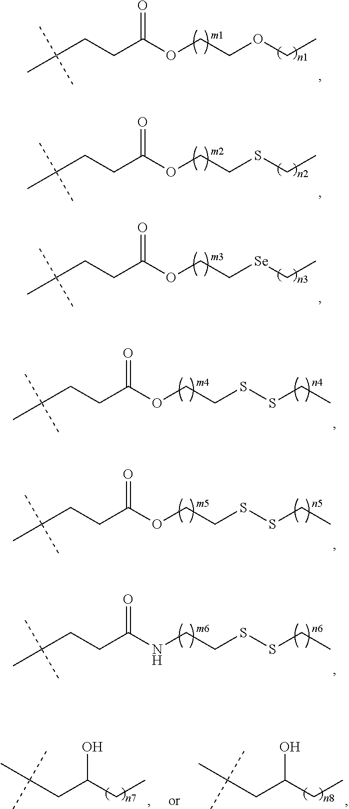

wherein the dashed line represents a connection (e.g., a bond) to the amine head, and wherein:
m1 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m2 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m3 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m4 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m5 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m6 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n1 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n2 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n3 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n4 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n5 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n6 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n7 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15); and
n8 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15).

In certain embodiments, each hydrophobic tail independently consists essentially of

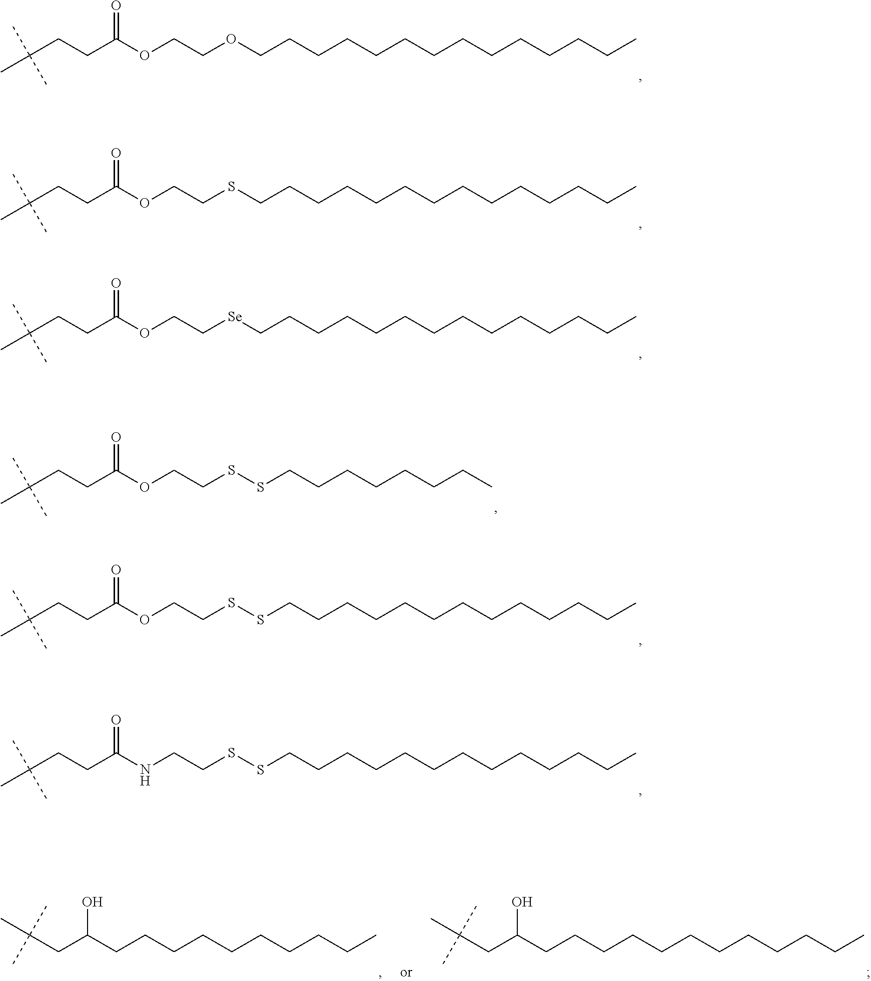

and
the dashed line represents a connection (e.g., a bond) to the amine head.

In certain embodiments, each hydrophobic tail consists essentially of

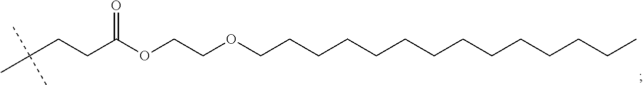

and
the dashed line represents a connection (e.g., a bond) to the amine head.

In certain embodiments, the alkyl chain is substituted with halo (e.g., fluoro).

In certain embodiments, each hydrophobic tail consists essentially of

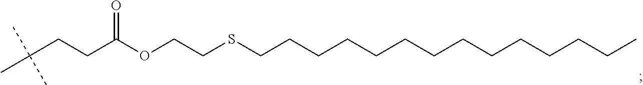

and
the dashed line represents a connection (e.g., a bond) to the amine head.

In certain embodiments, the alkyl chain is substituted with halo (e.g., fluoro).

In certain embodiments, the plurality of lipidoids forms a bilayer.

In certain embodiments, the immune modulator stimulates innate immunity. In certain embodiments, the immune modulator is capable of stimulating one or more immune-related genes. In certain embodiments, the immune modulator is capable of inhibiting one or more immune-related genes. In certain embodiments, the immune modulator is an immune enhancer. In certain embodiments, the immune modulator is an immune inhibitor. In certain embodiments, the immune modulator is a stimulator of interferon genes (STING). In certain embodiments, the STING is a STING agonist. In certain embodiments, the STING agonist is a cyclic dinucleotide. In certain embodiments, the STING agonist is cyclic guanosine monophosphate-adenosine monophosphate (cGAMP).

In certain embodiments, the immune modulator is encapsulated within the lipidoid composition. In certain embodiments, the plurality of lipidoids forms a bilayer and the immune modulator is encapsulated within the bilayer.

In certain embodiments, the lipidoid composition comprises an adjuvant. In certain embodiments, the adjuvant is a stimulator of the immune system. In certain embodiments, the stimulator of the immune system stimulates innate immunity. In certain embodiments, the stimulator of the immune system is a stimulator of interferon genes (STING). In certain embodiments, the STING is a STING agonist. In certain embodiments, the STING agonist is a cyclic dinucleotide. In certain embodiments, the STING agonist is cyclic guanosine monophosphate-adenosine monophosphate (cGAMP).

In certain embodiments, the adjuvant is encapsulated within the lipidoid composition. In certain embodiments, the plurality of lipidoids forms a bilayer and the adjuvant is encapsulated within the bilayer.

In certain embodiments, the lipidoid composition comprises an antigen. In certain embodiments, the antigen is a vaccine. In certain embodiments, the antigen is a protein. In certain embodiments, the antigen is an attenuated virus.

In certain embodiments, the antigen is encapsulated within the lipidoid composition. In certain embodiments, the plurality of lipidoids forms a bilayer and the antigen is encapsulated within the bilayer.

In certain embodiments, the lipidoid composition comprises a nucleic acid. In certain embodiments, the nucleic acid is a DNA or a RNA. In certain embodiments, the nucleic acid is an RNA. In certain embodiments, the RNA is an mRNA.

In certain embodiments, when the mRNA contacts a cell, the mRNA induces the synthesis of a protein belonging to a cancer cell. In certain embodiments, the cancer cell is a bladder cancer cell, breast cancer cell, brain cancer cell, bone cancer cell, cervical cancer cell, colorectal cancer cell, head cancer cell, neck cancer cell, kidney cancer cell, liver cancer cell, lung cancer cell, lymphoma cell, mesothelioma cell, myeloma cell, prostate cancer cell, skin cancer cell, thyroid cancer cell, ovarian cancer cell, or uterine cancer cell.

In certain embodiments, the mRNA contacts a cell, the mRNA induces the synthesis of a protein belonging to a virus. In certain embodiments, the virus is hepatitis C, norovirus, junin, dengue virus, coronavirus, human immunodeficiency virus, herpes simplex, avian flu, chickenpox, cold sores, common cold, glandular fever, influenza, measles, mumps, pharyngitis, pneumonia, rubella, severe acute respiratory syndrome, and lower or upper respiratory tract infection (e.g., respiratory syncytial virus). In certain embodiments, the virus is an influenza virus. In certain embodiments, the virus is a human immunodeficiency virus. In certain embodiments, the virus is a coronavirus. In certain embodiments, the coronavirus is SARS-CoV-2. In certain embodiments, the SARS-CoV-2 is the alpha, beta, gamma, delta, or omicron strain of SARS-CoV-2. In certain embodiments, when the mRNA contacts a cell, the mRNA induces the synthesis of the spike protein of the SARS-CoV-2.

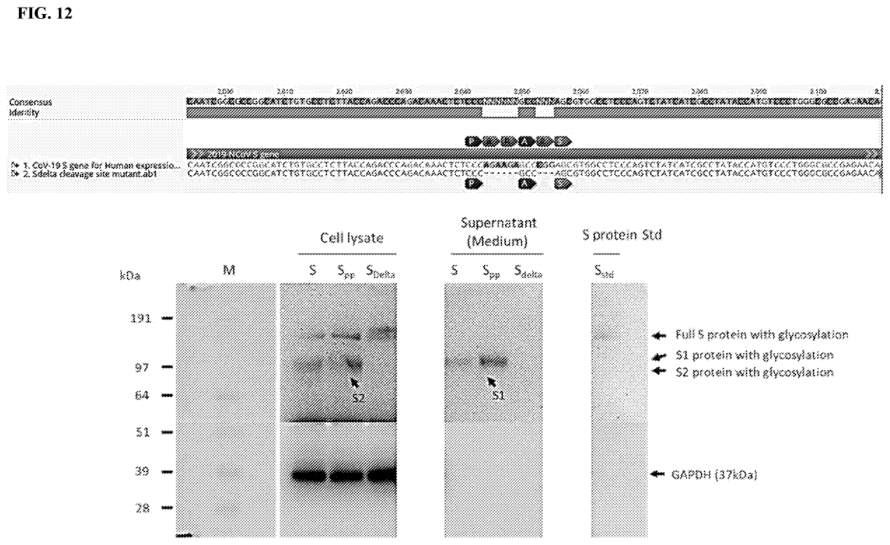

In certain embodiments, the mRNA has at least 60%, 65%, 70%, 75%, 80%, 85%, 90%, 95%, or 99% sequence identity to a sequence depicted in FIG. 12. In certain embodiments, the mRNA has at least 75%, 80%, 85%, 90%, 95%, or 99% sequence identity to a sequence depicted in FIG. 12. In certain embodiments, the mRNA has at least 90%, 95%, or 99% sequence identity to a sequence depicted in FIG. 12. In certain embodiments, the mRNA has at least 95%, or 99% sequence identity to a sequence depicted in FIG. 12. In certain embodiments, the mRNA has a sequence according to a sequence depicted in FIG. 12.

In certain embodiments, the lipidoid composition further comprises a chemotherapeutic agent. In certain embodiments, the chemotherapeutic agent is cytotoxic. In certain embodiments, the chemotherapeutic agent is an alkylating agent, an antimetabolite, an anti-microtubule agent, anti-tumor antibiotic, or a topoisomerase inhibitor, a mitotic inhibitor, or a corticosteroid. In certain embodiments, the chemotherapeutic agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-Fu), 6-mercaptopurine (6-MP), capecitabine, cladribine, clofarabine, cytarabine (Ara-C), decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, doxorubicin liposomal, epirubicin, idarubicin, valrubicin, bleomycin, dactinomycin, mitomycin-c, mitoxantrone, irinotecan, irinotecan liposomal, topotecan, etoposide, mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, or vorinostat. In certain embodiments, the chemotherapeutic agent is doxorubicin.

In certain embodiments, the lipidoid compositions are assembled with an (endogenous or exogenous) antigen, an immune modulator, an adjuvant, or any combination thereof. In certain embodiments, the lipidoid compositions are assembled with an (endogenous or exogenous) antigen. In certain embodiments, the lipidoid compositions are assembled with an immune modulator. In certain embodiments, the lipidoid compositions are assembled with an (endogenous or exogenous) antigen, and an immune modulator. In certain embodiments, the lipidoid compositions are assembled with an adjuvant.

In certain embodiments, the lipidoid compositions are capable of internalizing an antigen (e.g., an antigen from a tumor cell).

In another aspect, the present disclosure provides pharmaceutical composition comprising a lipidoid composition disclosed herein and a pharmaceutically acceptable excipient.

In another aspect, the present disclosure provides methods of treating cancer in a subject in need thereof comprising administering a therapeutically effective amount of a lipidoid composition disclosed herein.

In certain embodiments, the lipidoid composition is administered locally. In certain embodiments, the lipidoid composition is administered systemically. In certain embodiments, the lipidoid composition is administered intratumorally, intradermally, or intramuscularly. In certain embodiments, the lipidoid composition is administered intratumorally. In certain embodiments, the lipidoid composition is administered intradermally. In certain embodiments, the lipidoid composition is administered intramuscularly.

In certain embodiments, the lipidoid compositions are capable of internalizing an antigen (e.g., an antigen from a tumor cell).

In another aspect, the present disclosure provides pharmaceutical composition comprising a lipidoid composition disclosed herein and a pharmaceutically acceptable excipient.

In another aspect, the present disclosure provides methods of treating cancer in a subject in need thereof comprising administering a therapeutically effective amount of a lipidoid composition disclosed herein.

In certain embodiments, the lipidoid composition is administered intratumorally.

In certain embodiments, the lipidoid composition is administered locally. In certain embodiments, the lipidoid composition is administered systemically. In certain embodiments, the lipidoid composition is administered intratumorally, intradermally, or intramuscularly. In certain embodiments, the lipidoid composition is administered intratumorally. In certain embodiments, the lipidoid composition is administered intradermally. In certain embodiments, the lipidoid composition is administered intramuscularly.

In certain embodiments, the cancer is a solid tumor. In certain embodiments, the cancer is bladder cancer, breast cancer, brain cancer, bone cancer, cervical cancer, colorectal cancer, head cancer, neck cancer, kidney cancer, liver cancer, lung cancer, lymphoma, mesothelioma, myeloma, prostate cancer, skin cancer, thyroid cancer, ovarian cancer, or uterine cancer.

In certain embodiments, the method elicits an anti-cancer immune response in the subject.

In another aspect, the present disclosure provides methods of treating cancer in a subject in need thereof comprising the steps of administering a therapeutically effective amount of a chemotherapeutic agent to the subject; and administering a therapeutically effective amount of a lipidoid composition disclosed
herein to the subject.

In certain embodiments, the chemotherapeutic agent is cytotoxic.

In certain embodiments, the chemotherapeutic agent is an alkylating agent, an antimetabolite, an anti-microtubule agent, anti-tumor antibiotic, or a topoisomerase inhibitor, a mitotic inhibitor, or a corticosteroid.

In certain embodiments, the chemotherapeutic agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-Fu), 6-mercaptopurine (6-MP), capecitabine, cladribine, clofarabine, cytarabine (Ara-C), decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, doxorubicin liposomal, epirubicin, idarubicin, valrubicin, bleomycin, dactinomycin, mitomycin-c, mitoxantrone, irinotecan, irinotecan liposomal, topotecan, etoposide, mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, or vorinostat.

In certain embodiments, the chemotherapeutic agent is doxorubicin.

In certain embodiments, the chemotherapeutic is administered locally. In certain embodiments, the chemotherapeutic is administered systemically. In certain embodiments, the chemotherapeutic is administered intratumorally.

In certain embodiments, the chemotherapeutic is administered locally. In certain embodiments, the chemotherapeutic is administered systemically. In certain embodiments, the chemotherapeutic is administered intratumorally.

In certain embodiments, the lipidoid composition is administered about 6-48 hours after the chemotherapeutic agent. In certain embodiments, the lipidoid composition is administered about 6-24 hours after the chemotherapeutic agent. In certain embodiments, the lipidoid composition is administered about 24 hours after the chemotherapeutic agent.

In certain embodiments, the cancer is a solid tumor. In certain embodiments, the cancer is bladder cancer, breast cancer, brain cancer, bone cancer, cervical cancer, colorectal cancer, head cancer, neck cancer, kidney cancer, liver cancer, lung cancer, lymphoma, mesothelioma, myeloma, prostate cancer, skin cancer, thyroid cancer, ovarian cancer, or uterine cancer.

In certain embodiments, the method elicits an anti-cancer immune response in the subject.

In another aspect, the present disclosure provides methods of treating or preventing a viral infection in a subject in need thereof comprising administering a therapeutically effective amount of a lipidoid composition disclosed herein to the subject.

In certain embodiments, the method treats the viral infection. In certain embodiments, the method prevents the viral infection.

In certain embodiments, the viral infection is hepatitis C, norovirus, junin, dengue virus, coronavirus, human immunodeficiency virus, herpes simplex, avian flu, chickenpox, cold sores, common cold, glandular fever, influenza, measles, mumps, pharyngitis, pneumonia, rubella, severe acute respiratory syndrome, and lower or upper respiratory tract infection (e.g., respiratory syncytial virus). In certain embodiments, the viral infection is an influenza virus. In certain embodiments, the viral infection is a human immunodeficiency virus. In certain embodiments, the viral infection is a coronavirus. In certain embodiments, the coronavirus is SARS-CoV-2. In certain embodiments, the SARS-CoV-2 is the alpha, beta, gamma, delta, or omicron strain of SARS-CoV-2.

In certain embodiments, the method elicits an immune response in the subject. In certain embodiments, the method elicits an antiviral immune response in the subject.

In certain embodiments, the method vaccinates the subject against the viral infection.

In another aspect, the present disclosure provides kits comprising chemotherapeutic agent and a lipidoid composition disclosed herein.

In certain embodiments, the chemotherapeutic agent is cytotoxic.

In certain embodiments, the chemotherapeutic agent is an alkylating agent, an antimetabolite, an anti-microtubule agent, anti-tumor antibiotic, or a topoisomerase inhibitor, a mitotic inhibitor, or a corticosteroid.

In certain embodiments, the chemotherapeutic agent is the chemotherapeutic agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-Fu), 6-mercaptopurine (6-MP), capecitabine, cladribine, clofarabine, cytarabine (Ara-C), decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, doxorubicin liposomal, epirubicin, idarubicin, valrubicin, bleomycin, dactinomycin, mitomycin-c, mitoxantrone, irinotecan, irinotecan liposomal, topotecan, etoposide, mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, or vorinostat.

In certain embodiments, the chemotherapeutic agent is doxorubicin.

In another aspect, the present disclosure provides kits comprising chemotherapeutic agent and a pharmaceutical composition comprising a lipidoid composition disclosed herein.

In certain embodiments, the chemotherapeutic agent is cytotoxic.

In certain embodiments, the chemotherapeutic agent is an alkylating agent, an antimetabolite, an anti-microtubule agent, anti-tumor antibiotic, or a topoisomerase inhibitor, a mitotic inhibitor, or a corticosteroid.

In certain embodiments, the chemotherapeutic agent is the chemotherapeutic agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-Fu), 6-mercaptopurine (6-MP), capecitabine, cladribine, clofarabine, cytarabine (Ara-C), decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, doxorubicin liposomal, epirubicin, idarubicin, valrubicin, bleomycin, dactinomycin, mitomycin-c, mitoxantrone, irinotecan, irinotecan liposomal, topotecan, etoposide, mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, or vorinostat.

In certain embodiments, the chemotherapeutic agent is doxorubicin.

### Pharmaceutical Compositions

The compositions and methods of the present invention may be utilized to treat an individual in need thereof. The pharmaceutical composition described herein may comprise a therapeutic or prophylactic composition, or any combination thereof. In certain embodiments, the lipidoid compositions may be assembled with an antigen, an immune modulator, or any combination thereof. In certain embodiments, the individual is a mammal such as a human, or a non-human mammal. When administered to an animal, such as a human, the composition or the lipidoid composition is preferably administered as a pharmaceutical composition comprising, for example, a lipidoid composition of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a lipidoid composition such as a lipidoid composition of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a lipidoid composition of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those lipidoid compositions, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The lipidoid composition may also be formulated for inhalation. In certain embodiments, a lipidoid composition may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the lipidoid composition which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active composition, such as a lipidoid (e.g., nanoparticle) composition as described herein, with the carrier and, optionally, one or more accessory ingredients. In general, the formulations are prepared by uniformly and intimately bringing into association a lipidoid (e.g., nanoparticle) composition as described herein with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a lipidoid (e.g., nanoparticle) composition as described herein of the present invention as an active ingredient. Lipidoid compositions may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium lipidoid compositions; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered lipidoid composition moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active lipidoid compositions, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active lipidoid composition may be mixed under sterile conditions with a pharmaceutically acceptable carrier, and with any preservatives, buffers, or propellants that may be required.

The ointments, pastes, creams and gels may contain, in addition to an active lipidoid composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active lipidoid composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a lipidoid composition of the present invention to the body. Such dosage forms can be made by dissolving or dispersing the active lipidoid composition in the proper medium. Absorption enhancers can also be used to increase the flux of the lipidoid composition across the skin. The rate of such flux can be controlled by either providing a rate controlling membrane or dispersing the lipidoid composition in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active lipidoid compositions in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject lipidoid compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active lipidoid compositions can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a lipidoid composition at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular lipidoid composition or combination of lipidoid compositions employed, or the ester, salt or amide thereof, the route of administration, the time of administration, the rate of excretion of the particular lipidoid composition(s) being employed, the duration of the treatment, other drugs, lipidoid compositions and/or materials used in combination with the particular lipidoid composition(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or lipidoid composition at levels lower than that required in order to achieve the desired therapeutic effect and gradually increase the dosage until the desired effect is achieved. By “therapeutically effective amount” is meant the concentration of a lipidoid composition that is sufficient to elicit the desired therapeutic effect. It is generally understood that the effective amount of the lipidoid composition will vary according to the weight, sex, age, and medical history of the subject. Other factors which influence the effective amount may include, but are not limited to, the severity of the patient's condition, the disorder being treated, the stability of the lipidoid composition, and, if desired, another type of therapeutic agent being administered with the lipidoid composition of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active lipidoid composition used in the compositions and methods of the invention will be that amount of the lipidoid composition that is the lowest dose effective to produce a therapeutic effect. Such an effective dose will generally depend upon the factors described above.

If desired, the effective daily dose of the active lipidoid composition may be administered as one, two, three, four, five, six or more sub-doses administered separately at appropriate intervals throughout the day, optionally, in unit dosage forms. In certain embodiments of the present invention, the active lipidoid composition may be administered two or three times daily. In preferred embodiments, the active lipidoid composition will be administered once daily.

The patient receiving this treatment is any animal in need, including primates, in particular humans; and other mammals such as equines, cattle, swine, sheep, cats, and dogs; poultry; and pets in general.

In certain embodiments, lipidoid compositions of the invention may be used alone or conjointly administered with another type of therapeutic agent.

The present disclosure includes the use of pharmaceutically acceptable salts of lipidoid compositions of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

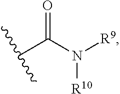

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

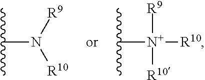

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

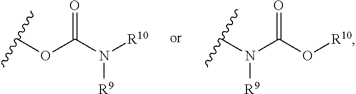

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

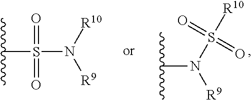

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

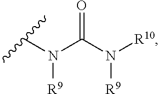

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds disclosed herein. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds disclosed herein. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the lipidoid compositions (e.g., nanoparticles) useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Some of the lipidoid compositions (e.g., nanoparticles) may also comprise chemical compound which exist in tautomeric forms. Such forms, although not explicitly indicated in the formulae described herein, are intended to be included within the scope of the present disclosure.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

### EXAMPLES

The invention now being generally described, it will be more readily understood by reference to the following examples which are included merely for purposes of illustration of certain aspects and embodiments of the present invention, and are not intended to limit the invention.

### Example 1: Preparation of Exemplary Vaccines

### Summary

In situ vaccination is a promising strategy for cancer immunotherapy owing to its convenience and the ability to induce numerous tumor antigens. However, the advances of in situ vaccination is also hindered by low cross-presentation of tumor antigens and the immunosuppressive tumor microenvironment. To balance the safety and efficiency of in situ vaccination, a lipidoid nanoparticle (LNP) was designed for enhanced cross-presentation and STING activation. After the library screening, 93-O17S-F was shown to promote both the cross-presentation of tumor antigens and the delivery of cGAMP (STING agonist). Intratumorally injection of 93-O17S-F/cGAMP in combination of pre-treatment of doxorubicin exhibited excellent antitumor efficacy with 35% of the total recovery rate of primary B16F10 tumor and 71.2% of free-of-tumor rate of rechallenging B16F10 tumor.

### DISCUSSION

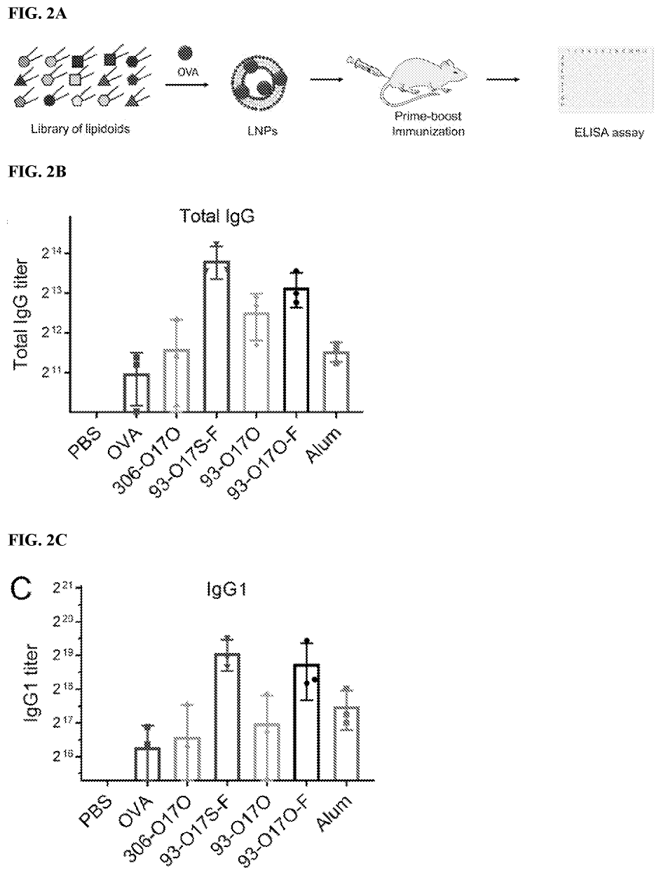

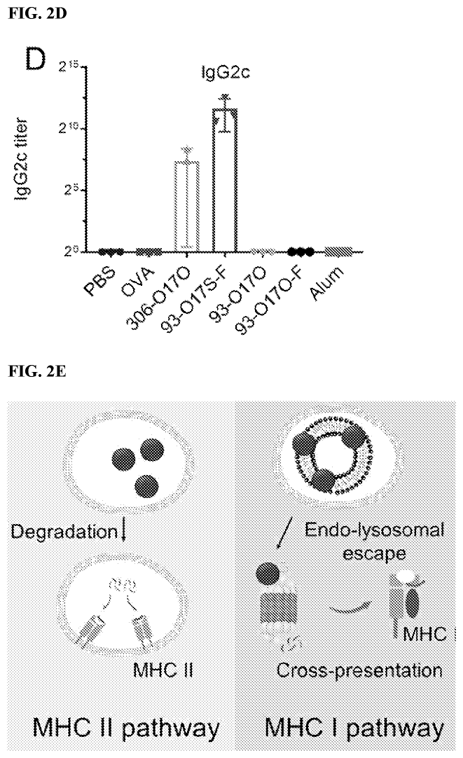

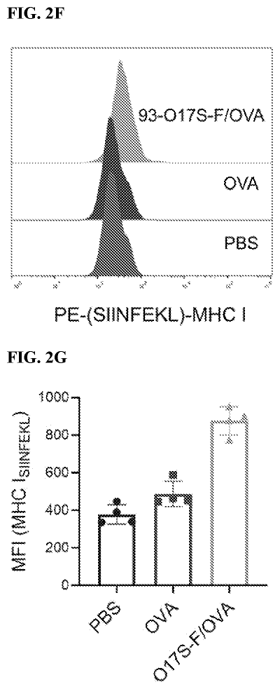

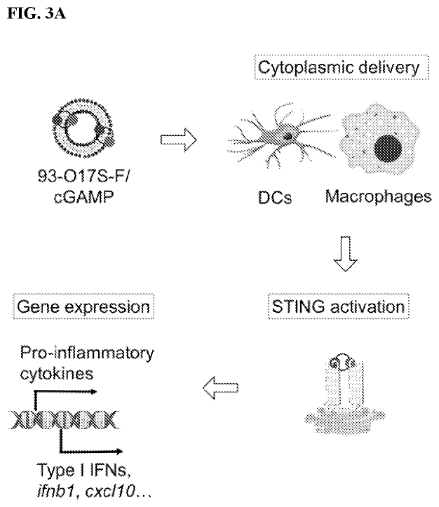

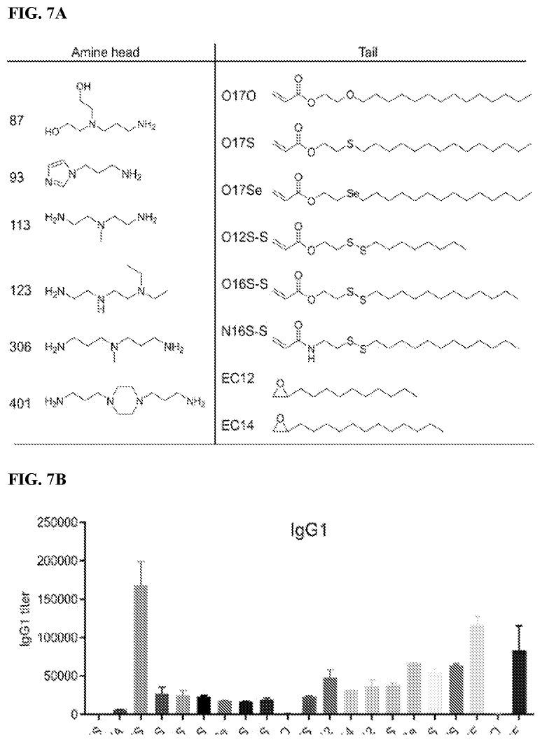

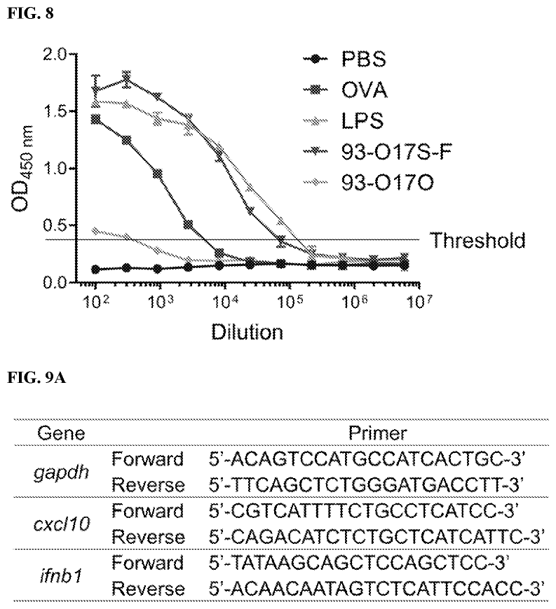

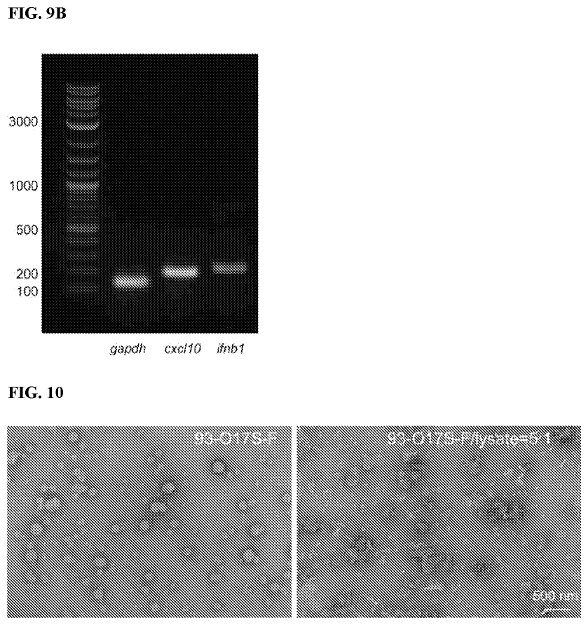

The composition and structure of lipidoids greatly influence the adjuvant effect of the LNPs, including the antigen delivery and immunostimulation. Ac ombinatorial library strategy has been used to develop synthetic lipidoids with various structures and properties for drug delivery. An ideal lipid nanoparticle for cancer immunotherapy should be able to i) capture the released tumor antigen and deliver into antigen presentation cells with enhanced cross-presentation, and ii) generate immunostimulatory effect. To identify the effective lipidoids, a rough screening of a selected library was carried out by evaluating the antibody response in C₅₇BL/6 mice after immunization with the model antigen ovalbumin (OVA) formulated with different LNPs (FIG. 1A). 18 different lipidoids with varied heads and tails were formulated with OVA separately (FIG. 7A). Mice were immunized via a prime-boost immunization strategy with two injection at day 1 and day 14, respectively. As shown in FIG. 7B, lipopolysaccharide (LPS) was used as the positive control and showed extremely positive immunoglobulin G 1 (IgG1) antibody response. However, OVA alone without formulation had very low antibody response due to its low immunogenicity. Interestingly, two of the lipidoids with 93 amine head in the selected library, 93-O17S-F and 93-O170-F, showed comparable antibody response as LPS. The detailed IgG1 and IgG2c antibody response of these lipidoids was further evaluated. IgG1 antibody was mainly induced by CD4⁺ T helper type 2 (Th2) cells, while IgG2c antibody was generated by CD4⁺ T helper type 1 (Th1) cells.²⁴ Th1 cells could activate macrophages and produce memory T cells, which is important for the killing of tumor cells. As shown in FIGS. 2B & 2C, the total IgG and IgG1 response was consistent with the result from rough screening.

The three leading LNPs showed much stronger antibody response than OVA alone, as well as the US FDA approved adjuvant Alum. However, only the 93-O17S-F generated high IgG2c response, representing the activation of Th1 cells (FIG. 2D). Moreover, the excipients in the LNP formulation also greatly affected the antibody production. Without the helper lipids including cholesterol and dioleoylphosphatidylethanolamine (DOPE), the 93-O17S showed no antibody response at all. In short, 93-O17S formulated with excipients (93-O17S-F) is able to induce robust Th1 and Th2 responses with much better efficacy than the established Alum adjuvant.

Apart from the successful activation of both CD4⁺ Th1 and Th2 cells by 93-O17S-F, the LNPs also serve as delivery vehicle to deliver tumor antigen intracellularly to enhance activation of CD8⁺ T cell via cross-presentation. Free antigen, such as OVA is degraded by enzymes in lysosome and then bound to MHC II molecule, leading to mainly antibody-based immune response. When the antigens delivered into cytosol can be degraded by proteasome and the antigen derived peptide can be bound to MHC I molecules, then enhances the cross-presentation to CD8⁺ T cells which is known to be more important in fighting cancers. Model OVA antigen with 93-O17S-F was delvied to cells and then the cells were stained with anti-mouse H-2Kb bound to SIINFEKL antibody after incubation for 24 h. The DC2.4 cells treated with free OVA was used as control. As shown in FIGS. 2G & 2F, the shift of the fluorescence was observed in the 93-O17S-F/OVA treated cells compared with free OVA-treated cells. The mean fluorescence intensity (MFI) calculated by the flow cytometry confirmed the enhanced expression of SIINFEKL-MHC I complexes in 93-O17S-F/OVA treated cells, which is about 1.8 times of that in free OVA groups. These results showed the enhanced cross-presentation of antigen (OVA) delivered in 93-O17S-F.

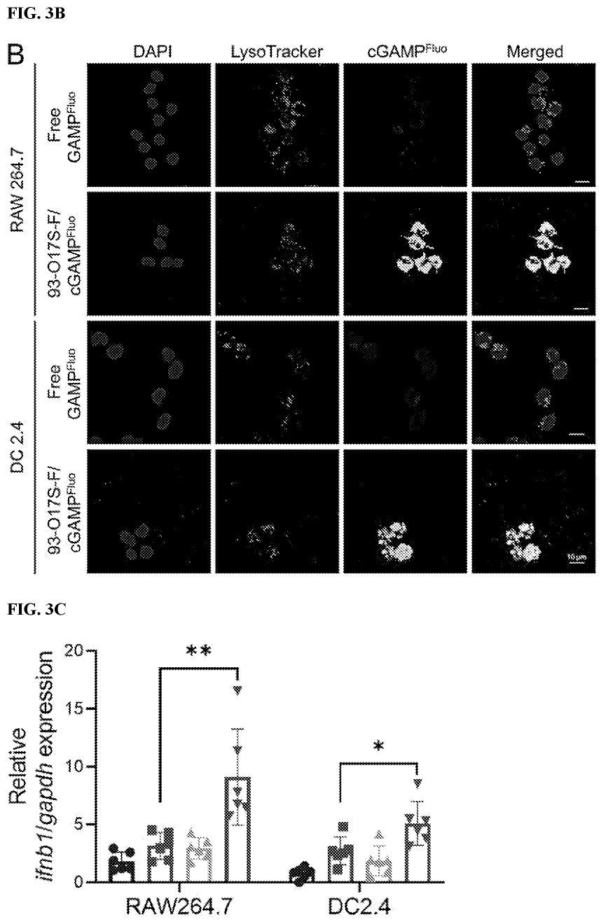

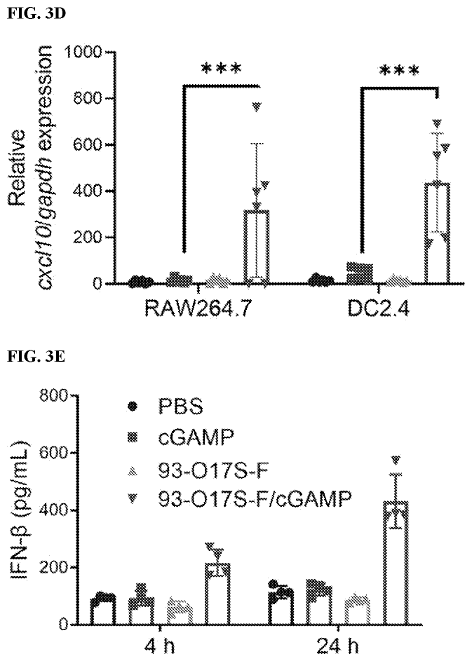

In order to further enhance the immune stimulation for cancer immunotherapy, cGAMP, an agonist for STING pathway, was chosen to be encapsulated in the 93-O17S-F for intracellular delivery. The recognition of cGAMP by STING resulted in the activation of APCs, the production of IFNs, and the priming of CD8⁺ T cells against tumor antigens, which was proved to be critically important in caner immunotherapy.^{26,27} However, cGAMP itself is not able to freely cross cell membrane to reach the STING promoters on endoplasmic reticulum. it was hypothesized that the lipidoid nanoparticles such as 93-O17S-F, can serve the carrier for cGAMP through electrostatic interaction and facilitate its intracellular delivery to activate STING pathway (FIG. 3A). To prove the hypothesis, the intracellular distribution of cGAMP was evaluated by confocal laser scanning microscopy (CLSM) using the fluoresceinyl-labelled cGAMP (cGAMP^{Fluo}). The cGAMP^{Fluo} was encapsulated into 93-O17S-F by simple mixing and then added into the medium of RAW264.7 and DC2.4 at the dose equivalent to 200 ng/mL cGAMP^{Fluo}. FIG. 3B showed the enhanced endocytosis and endo/lysosome escape of cGAMP delivered by 93-O17S-F. After 4 h of incubation, the cGAMP^{Fluo} encapsulated in 93-O17S-F were observed as strong green signal in both RAW264.7 and DC2.4 cells. However, there is almost no green signal of the cGAMP^{Fluo} in the cells treated by free cGAMP^{Fluo 0} wing to the low cell membrane permeability. More importantly, FIG. 3B showed the cGAMP could escape from the endo/lysosome into the cytoplasm of both RAW264.7 and DC2.4 cells. The release of cGAMP is crucial for the following activation of STING located in endoplasmic reticulum.

To evaluate the promoted STING activation by the cytoplasmic delivery of our LNPs, the expression of ifnb1 and cxcl10 genes was measured by real time polymerase chain reaction (RT PCR) in RAW264.7 and DC2.4 cells. The ifnb1 and cxc110 genes are two of the main genes related to the activation of STING, resulting in abundant type I IFNs and pro-inflammatory cytokines secretion. As shown in FIG. 3C, the expressions of ifnb1 genes in both RAW264.7 and DC2.4 cells treated with 93-O17S-F/cGAMP were about 6.9 and 6.4 folds compared with that of the cells treated with PBS. Free cGAMP only showed modest increase of the expression of ifnb1 due to its low penetration of cell membrane. The similar trend was also observed in the expression of cxcl10 gene in FIG. 3D. The 93-O17S-F/cGAMP generated dramatically increased expression (more than 100 folds) of cxc10 gene, further confirming the activation of STING pathway. Finally, the secretion of IFN-β, a typical type I interferon, was measured after the treatment of different cGAMP formulations in FIG. 3E. In consistent to gene expression, the 93-O17S-F/cGAMP induced higher concentration of secreted IFN-β. The secretion of IFN-β kept increasing even till 24 h, while the concentration of IFN-β in other groups almost unchanged. The upregulated expression of STING-activation related genes and pro-inflammatory factors proved that the 93-O17S-F could enhance the activation of STING pathway via promoted cytoplasmic delivery.

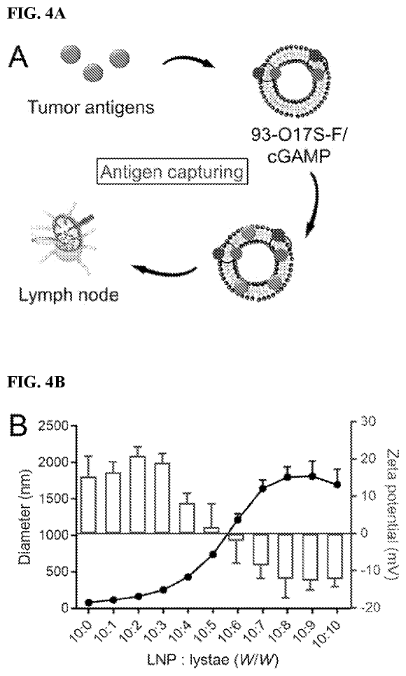

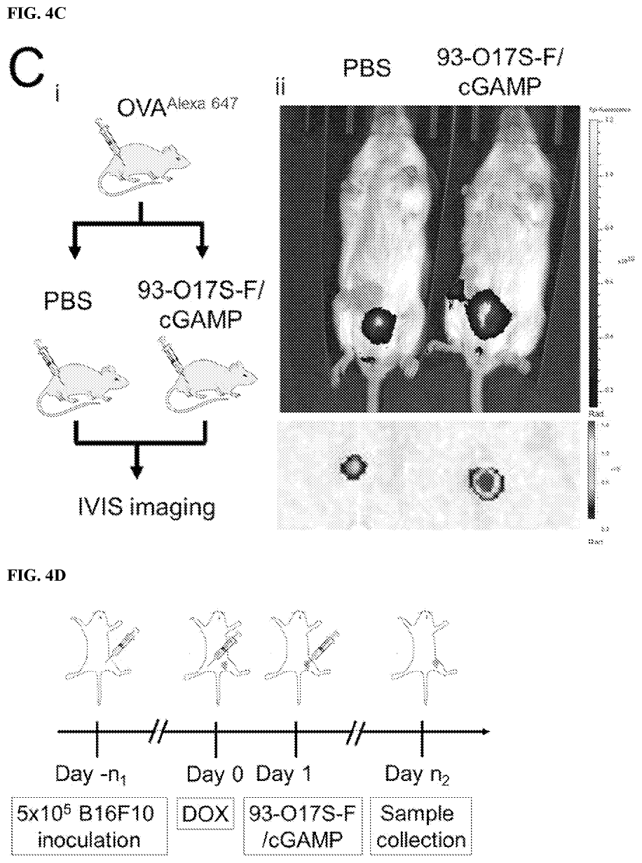

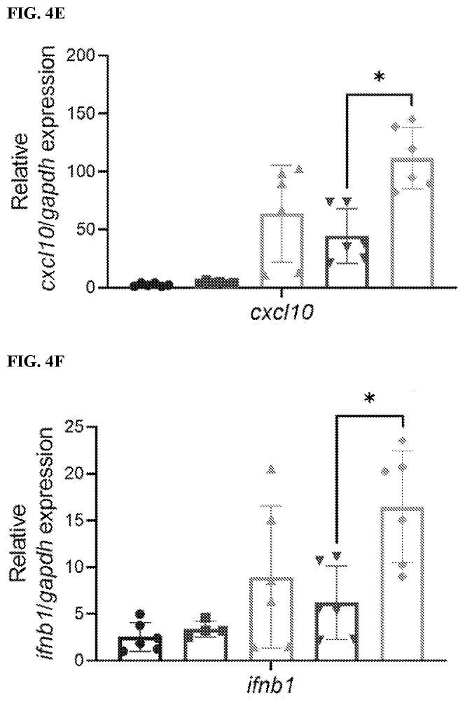

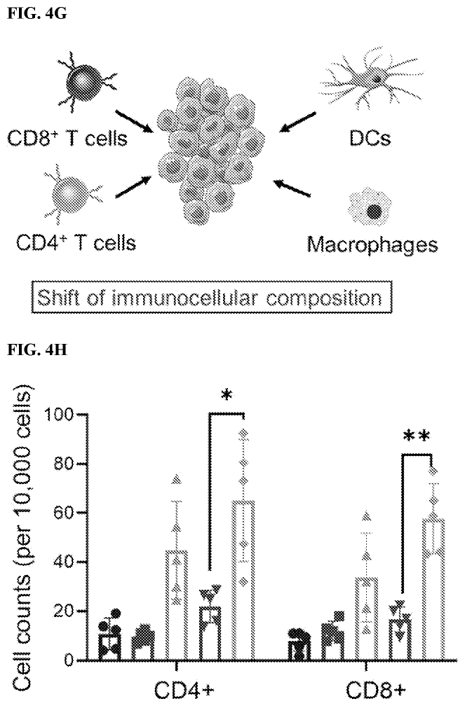

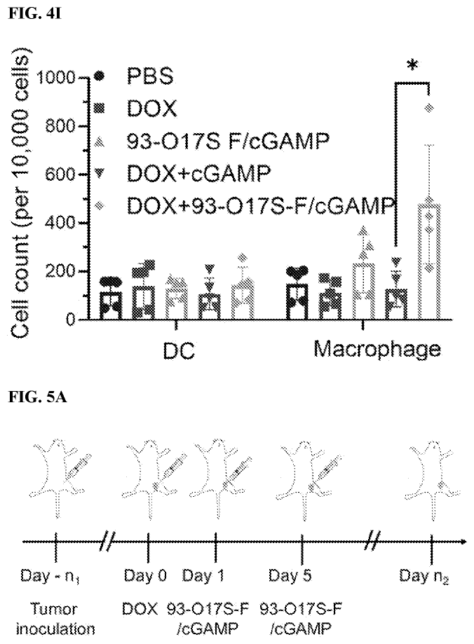

One of the distinguishable advantages of this design is that the LNP can also capture and deliver the tumor antigens released from the tumor cells after the treatment of small amount of chemotherapeutics (FIG. 4A). The antigen capture ability was initially investigated through the changes of size and zeta potential of the LNP after incubation with tumor lysates. As shown in FIG. 4B, the size of the LNP-tumor lysate complex increased dramatically with the increasing of the weight ratio of LNP to tumor lysate due to the absorption or aggregation of the complex. When the weight ratio increased to 1:0.6, the size reached the maximum value due to the limited capacity of LNP. FIG. 10 showed the transmission electron microscopy images of blanked 93-S14-F alone or complexed with tumor lysate. The blank 93-S14-F showed well-separated spherical morphology, while formed clusters after mixing with tumor lystates. It proved the interaction between the LNP and tumor lysates. Furthermore, the zeta potential of the 93-S14-F decreased from positive charge to negative charge with the increase of weight ratio of LNP to tumor lysate. This showed that the tumor lysate (including tumor antigen) capture is mediated by the electrostatic interaction.

In order to evaluate whether such in antigen capture and delivery to draining lymph node occurs in the complex in vivo environment, an in vivo experiment for antigen capture and delivery using the Alexa Fluor 647-conjugated OVA (OVA^{Alexa-647}) as a model antigen was developed. The OVA^{Alexa-647} was subcutaneously injected at right flank of mice, simulating the antigens released after the administration of DOX. Then 93-O17S-F/cGAMP or PBS control were injected at the same location with the OVA^{Alexa-647} for the capture of the released model antigen (FIG. 4C). After 5 h of the second injection, the mice were imaged using in vivo imaging system (IVIS). As shown in FIG. 3C-ii, free OVA^{Alexa-647} followed with PBS injection was mainly distributed in bladder, suggesting the rapid clearance of the soluble protein through urine. While the fluorescence signal of OVA^{Alexa-647} was observed in the draining lymph node in the mice treated with free OVA^{Alexa-647} followed with 93-O17S-F/cGAMP injection on the same spot. The draining lymph nodes of the two group were harvested and imaged (FIG. 4C). The fluorescence intensity of the draining lymph node from mouse treated with free OVA^{Alexa-647} followed with 93-O17S-F/cGAMP injection were much higher than that from mouse treated with free OVA^{Alexa-647} followed with PBS injection on the same spot. This result demonstrates that the LNP such as 93-O17S-F can capture free proteins in the tissue and carry them to the draining lymph node. Such capability is useful to capture the tumor antigens from tumor lysate and present to APCs in draining lymph node, which may enhance the antitumor effect and reduce the immune escape.

To summarize, the 93-O17S-F nanoparticle formulation is capable of capturing protein antigen, enhancing antigen cross-presentation, and simultaneously delivering STING agonist cGAMP. The in vivo STING activation was further evaluated using the LNP formulation in B16F10 subcutaneous tumor-bearing C57BL/6 mice, as illustrated in FIG. 4D. To generate tumor model, 5×10⁵ B16F10 cells were injected at the right flank of 4-6 weeks old C57BL/6 mice. The tumors were allowed to grow up to 60 to 80 mm³ and the mice were divided into five groups. At day 0, free DOX was injected into three groups of the tumors directly to induce immunogenetic death and release large amount of tumor associate antigens (TAAs). At day 1, solutions, including PBS, 93-O17S-F, free cGAMP, and formulated 93-O17S-F/cGAMP, were injected into the same site where DOX was injected, respectively. After 6 h post the second injection, the tumors were collected and the expression of ifnb1 and cxcl10 genes were analyzed by RT-PCR. As shown in FIGS. 4E & 4F, comparing with tumor treated with DOX only, the expression of both ifnb1 and cxcl10 did not changed significantly for tumor injected with PBS one day after the DOX injection. The tumor treated with free cGAMP one day after the DOX injection showed modest increase of these two genes, indicating in situ administration of STING agonist only activates the STING pathway modestly. However, the 93-O17S-F/cGAMP treated groups all showed increased expression of ifnb1 and cxcl10 genes compared with free cGAMP, regardless DOX was administrated before the second injection or not. The administration of DOX also enhanced the activation of STING pathway by 93-O17S-F/cGAMP to some extent, which might be due to the released tumor antigens.

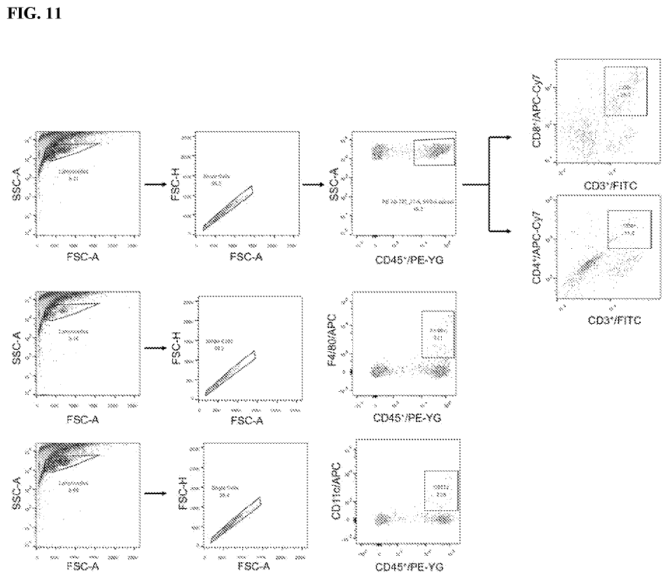

The successful activation of STING pathway by the LNP system could also change the immunocellular composition of the tumor microenvironment. As shown in FIG. 4G, the activation of STING pathway stimulated the secretion of pro-inflammatory factors including multiple chemokines that could recruit various immune cells to tumor sites. To further evaluate the changes of the cellular composition after the treatment, the tumors were collected at 48 h after the second injection. The tumors were dissociated to single cells, stained with antibodies, and analyzed by the flowcytometry (FIG. 11). The treatment of DOX alone did not make significant changes to the population of both CD4⁺ and CD8⁺ T cells (FIG. 4H). The free cGAMP treated tumors exhibited an increasement of both CD4⁺ and CD8⁺ T cells populations, indicating the administration of STING agonist could indeed make contribution to the immunotherapy of solid tumor. However, after encapsulation into 93-O17S-F, significant increase of T cell populations was observed in 93-O17S-F/cGAMP treated tumors, especially in the DOX-pretreated group. Moreover, the population of macrophages also showed similar trend with the population of T cells at tumor sites (FIG. 4I). However, the population of DCs showed no significant changes in any treated group. The recruitments of T cells and macrophages were related with the activation level of STING pathway, which could benefit the therapeutic outcome of this in situ vaccination strategy.

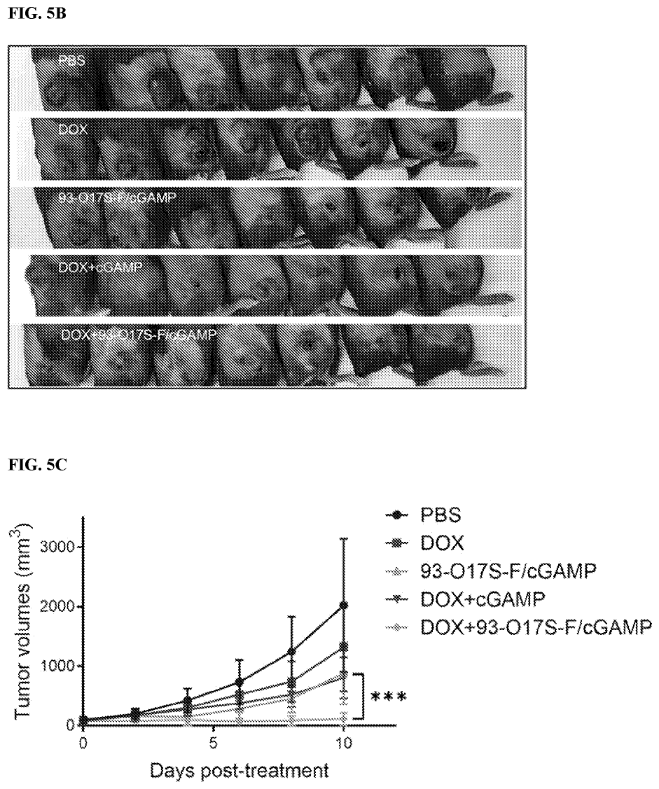

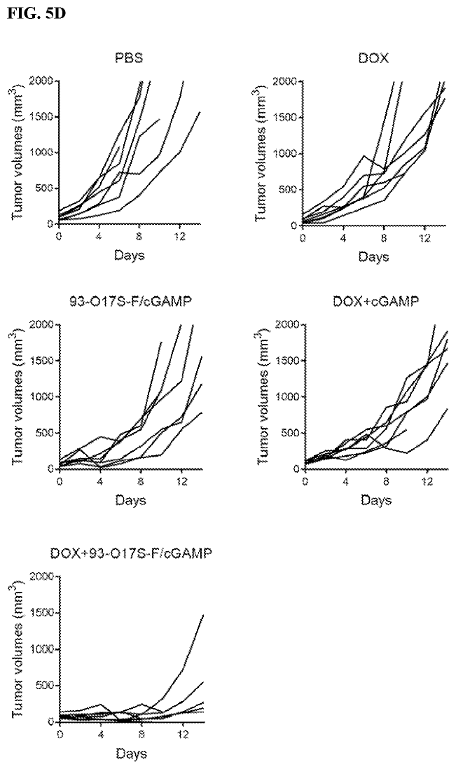

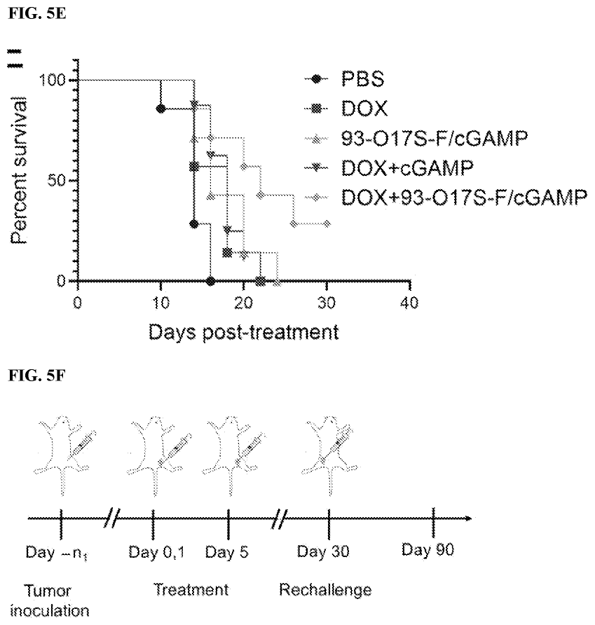

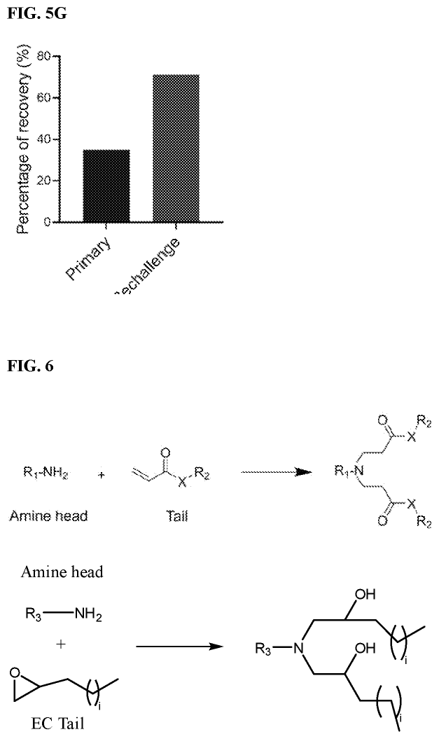

Finally, the antitumor effect of the LNP system was evaluated in B16F10 xenografted tumor model. As shown in FIG. 5A, B16F10 cells were injected subcutaneously into the right flank of the back. When the tumor grew to 60 to 80 mm³, the mice were divided into five groups including PBS, DOX, 93-O17S-F/cGAMP, DOX+cGAMP, and DOX+93-O17S-F/cGAMP. The groups of DOX, DOX+cGAMP, and DOX+93-O17S-F/cGAMP were treated with DOX at day 0, and then treated with PBS, free cGAMP, or 93-O17S-F/cGAMP at day 1 and 5 respectively. The images of tumors were captured at day 6 in FIG. 5B. Small ulcerations were observed on the tumors after treatment in every group, especially the DOX+93-O17S-F/cGAMP group. Among all experimental groups, the tumors in DOX+93-O17S-F/cGAMP group were the smallest, some with indivisible tumors after the treatment. In FIG. 5C, the curves of tumor volumes at the first 10 days were listed. The tumor treated with PBS still grew very rapidly and reach to the average size of about 2,000 mm³. The treatment of DOX only had slight inhibition of the tumor due to the induced apoptosis. The single administration of 93-O17S-F/cGAMP or free DOX+cGAMP inhibited the tumor to some extent but the tumors kept growing. The detailed tumor volumes of each mouse were shown in FIG. 5D. The tumor volumes of PBS-treated mice exceed 2,000 mm³ within 12 days. The free DOX only slightly postponed the rapid growth compared with that in PBS group while the tumor volumes still reached 2,000 mm³ soon. The treatment of 93-O17S-F/cGAMP without pre-treatment of DOX showed modest inhibition of tumor before day 8 but still cannot inhibit the rapid tumor growth for long time. The results proved that the 93-O17S-F/cGAMP could activate the STING pathway in tumor and inhibit the tumor growth to some extent. However, the long-term inhibition of tumors still be hindered by the insufficiently presented tumor antigens without the pre-induction of DOX. In DOX+93-O17S-F/cGAMP group, most of the tumor growth were inhibited and two of them even got free of tumor at the end of the experiment. The survival rate of mice during the treatment was shown in FIG. 5E. The mice treated with PBS all reached the humane endpoints within 14 days. The administration of free DOX, 93-O17S-F/cGAMP, or DOX+cGAMP only exhibited modest improvement of the survival rate and all the mice reached the humane endpoints within 25 days. The mice treated with DOX+93-O17S-F/cGAMP showed significantly extended survival rate and two of the seven mice get eradication of tumors within 30 days. The excellent antitumor efficacy of the LNP system further confirmed the superiority of 93-O17S-F/cGAMP for in situ vaccination owing to the enhanced cross-presentation and STING activation.

### Exemplary Experimental Details

### ELISA Assays

The plates were covered with 50 μL of OVA at 20 μg mL⁻¹ in sodium carbonate solution (pH 8.0) at 4° C. overnight. The plates were then washed by PBST (PBS with 0.5% tween-20) for three times and blocked by 5% bovine serum albumin solution (Sigma-Aldrich). The serum collected from immunized mice was diluted in triplicate from 1:100 and then added into the plates for 2 hours at RT. Then the plates were washed three times and incubated with 100 L of HRP-conjugated IgG, IgG1, and IgG2c antibodies (1:10,000 dilution) for another 2 hours. The plates were washed by PBST for three times and incubated with 100 μL of 3,3′,5,5′-tetramethylbenzidine (TMB) substrate. The reaction was stopped by 0.16 M sulfuric acid solution. The optical density (O.D.) values at 450 nm was read in BioTex microplate reader. As shown in Figure S3, the endpoint titer is defined as the reciprocal of the highest dilution of a serum that gives a reading above the cutoff.

### Detection of MHC I Complex Bound to SIINFEKL

5×10⁴ DC2.4 cells was cultured in 24 well plates for 24 hours. Then the cells were incubated with OVA or 93-O17S-F at doses equivalent to 1 μg mL^{-1 0} f OVA for 24 h. The cells were collected, washed with PBS, and stained with PE anti-mouse H-2Kb bound to SIINFEKL antibody in flow cytometry staining buffer (eBioscience) at 4° C. for 2 hours. The stained cells were washed by PBS and then detected by Attume NxT flow cytometer. The data was analyzed by FlowJo-v10.

Cellular Uptakes of cGAMP

5×10⁴ RAW264.7 and DC2.4 cells were cultured in the chambered coverslip with 4 wells. The free cGAMP^{Fluo} or 93-O17S-F/cGAMP^{fluo} was added to the medium at doses equivalent to 200 ng/mL cGAMP. After 4 h of incubation, the medium was replaced and incubated by fresh medium containing LysoTracker Red DND-99 for another 1 h. Then the cells were washed by PBS and fixed by 4% paraformaldehyde (PFA) solution for 10 min. Then the slices were cover by Fluoroshield™ with DAPI (Sigma-Aldrich). The images were capture by Leica TCS SP8 microscopes.

### In Vitro Evaluation of Ifnb1 and Cxcl10 Genes Expression by RT PCR

5×10⁵ RAW264.7 and DC2.4 cells were cultured in 6 well plates for 24 hours. Then the cells were treated with PBS, free cGAMP, 93-O17S-F, or 93-O17S-F/cGAMP for 4h at doses equivalent to 150 nM cGAMP. The total mRNA was isolated by TriPure reagent (Sigma-Aldrich) following the manufacturer's manual. The complementary DNA (cDNA) was synthesized using high-capacity cDNA reverse transcription kit (Applied Biosystems). The RT PCR was carried out using Applied Biosystems PowerUp™ SYBR™ green master mix and detected in BioRad CFX96 touch Real-Time PCR detection system. The relative gene expressions were analyzed by CFX maestro software.

### In Vivo Antigen Capture and Delivery

50 μL of OVA^{Alexa-647} at the concentration of 1 μg/L was injected subcutaneously into the right flank of BALB/c mice. 5 min after the first injection, 50 μL of PBS or 93-O17S-F/cGAMP containing 200 μg of LNP were injected subcutaneously at the location of first injection. The distribution of OVAAlexa-647 and the isolated draining lymph nodes were monitored by IVIS Spectrum in vivo imaging system.

### In Vivo Evaluation of Ifnb1 and Cxcl10 Genes Expression by RT PCR

5×10⁵ B16F10 cells were inoculated in the right flank of 4-6 weeks old C57BL/6 mice. The tumors were allowed to grow up to 60 to 80 mm³. Mice were divided into five groups including PBS, free DOX (noted as DOX), 93-O17S-F/cGAMP, free cGAMP post the administration of DOX (noted as DOX+cGAMP), and 93-O17S-F/cGAMP post the administration of DOX (noted as DOX+93-O17S-F/cGAMP). At day 0, DOX was intratumorally injected into three groups of the tumors directly at doses equivalent to 0.1 mg per kg body weight ((kg BW)⁻¹). At day 1, the second injection including PBS, 93-O17S-F LNP, free cGAMP, and formulated 93-O17S-F/cGAMP was intratumorally injected into the same site where DOX was injected at doses equivalent to 20 μg of cGAMP encapsulated 400 μg of 93-O17S-F LNP per mouse. After 6 h post the second injection, the tumors were collected, and the total mRNA were isolated using TriPure reagent. The detailed RT PCR was carried out in the same route as before.

### Modulation of Immunocellular Composition of the Tumor Microenvironment

The tumor model was built and treated as same as described in section 6. After 48 h post the second injection, the tumors were collected and suspended to single cell solutions by 40 μm strainer (Thermo-Fisher). The cells were collected, washed with PBS, and stained with fluorescent antibodies of CD4 (APC-Cy7), CD8a (PE-Cy5), CD45 (PE), CD3e (FITC), F4/80 (APC), and CD11c (APC) in Flow Cytometry Staining Buffer (eBioscience). Fluorescent signal was measured using LSR-II flow cytometer (BD Biosciences). The data was analyzed by FlowJo-v10.

### Treatment of B16F10 Tumor by In Situ Vaccination

The tumor model was built as same as described in section 6. Mice were also divided into five groups including PBS, DOX, 93-O17S-F/cGAMP, DOX+cGAMP, and DOX+93-O17S-F/cGAMP. At day 0, DOX was intratumorally injected into three groups of the tumors directly at doses equivalent to 0.1 mg per kg body weight ((kg BW)⁻¹). At day 1 and 5, the second and third injections including PBS, 93-O17S-F LNP, free cGAMP, and formulated 93-O17S-F/cGAMP was intratumorally injected into the same site where DOX was injected at doses equivalent to 20 μg of cGAMP encapsulated in 400 μg of 93-O17S-F LNP per mouse. The length (L) and width (W) of the tumors were measure every other day and the tumor volumes (V) were calculated by the equation: V=LxW²/2.

The experiment of tumor rechallenge was carried out as follows. 20 mice were treated as same as described before. The mice free of tumor for 30 days were re-inoculated with 2×10⁵ B16F10 cells and monitored up to 90 days.

### Example 2: Further Preparation of Exemplary Vaccines

### Summary

The rapid-spreading pandemic caused by severe acute respiratory syndrome coronavirus-2 (SARS-CoV-2) is inflicting enormous suffering and economic loss worldwide. There were more than 44 million reported cases and 1 million deaths in the world till the end of October 2020. To the date, 11 vaccines were involved in phase III trials. Among these vaccines, mRNA-based vaccines showed great advantages because it is built on precise but adaptable antigen design, induces both CTL and nAb responses, and avoids many of the drawbacks of other forms of vaccines (protein, inactivated virus, DNA and viral vectors). However, cytotoxic T cell responses of mRNA-1273 vaccine (Moderna Therapeutics) in nonhuman primates were still low. It was hypothesized the addition of adjuvants enhancing the CTL production would upregulate the cytotoxic T cell response of mRNA vaccine. A lipid library was screened for in vivo mRNA delivery and successfully found a candidate lipidoid which could deliver mRNA to macrophages and DCs in draining lymph nodes. The adjuvant Pam₃CSK₄ was then incorporated to the LNP and found the addition of Pam₃CSK₄ increased the expression of luciferase mRNA in draining lymph nodes. The evaluation of the vaccination by adjuvant-contained Lipid nanoparticles have been processed.

### DISCUSSION

To optimize selection of T cell and B cell epitopes and antigen designs.

CD4 and CD8 T cell epitopes have been computationally characterized and identified CD4 and CD8 T cell epitopes from SARS-CoV-2 using an ensemble of machine learning algorithms that consider conservation of viral sequences, expression level, glycosylation, structural constrains, MHC allelic distribution in the human population. The identified epitopes will be validated in vitro using peripheral blood mononuclear cells from SARS-CoV-2 convalescent individuals. The validated epitopes will be constructed into mRNA for optimal expression, processing and presentation.

Three kinds of mRNAs candidates have been identified for vaccine design. 1. Full spike protein (S). 2. Full spike protein with two proline mutation. (Sₚₚ) 3. Full spike protein with mutated furin cleavage site. (S_{delta}).

To deliver vaccine mRNA into dendritic cells in the draining lymph nodes using novel synthetic lipid nanoparticles.

A new class of imidazole containing lipids that target mRNA to DCs or draining lymph nodes have been identified. Encoding mRNA will be formulated into lipid nanoparticles (LNP) (mRNA-LNPs) using these new lipids and other known lipids and compare their delivery of mRNA into DCs in the draining lymph nodes and induction of CTL and nAb responses in HLA-A2 transgenic mice. The targeted delivery and immune responses will be optimized using DC-targeting ligands.

Lipidoid composition as described herein with enhanced mRNA expression in draining lymph nodes, as compared with the ALC-0315 LNP used in Pfizer-biontech's BNT162b2 mRNA vaccine, will be produced.

To augment nAb response with Toll-like receptor (TLR) ligands.

TLR ligands (Pam₃CSK₄ and poly I:C) that dramatically augment nAb responses to a protein-based dengue virus vaccine while reducing non-neutralizing antibody responses have been identified. The two TLR ligands individually will be produced and in combination into mRNA-LNPs and test if they enhance nAb response and reduce antibody-dependent enhancement. The mechanisms underlying the enhanced nAb response will be investigated for further adjuvant optimization.

To determine whether select mRNA vaccines confer protection against SARS-CoV-2 infection in a small animal model.

ACE2 transgenic mice and hamster are susceptible to SARS-CoV infection. ACE2 transgenic mice or hamsters or ferrets will be vaccinated with the most optimized vaccine candidates and the induction of immune responses will be evaluated by ELISA, SARS-CoV-2 neutralization and CTL assays. Vaccinated animals will be challenged with SARS-CoV-2. Clinical signs, viral load and animal health status will be monitored. Together, these data will permit an evaluation of vaccine efficacy in the mitigation of SARS-CoV-2 in a relevant pre-clinical animal model.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A lipidoid composition (e.g., a lipidoid nanoparticle composition for delivery to immune cells) comprising a plurality of lipidoids; and an adjuvant, an antigen, or a nucleic acid.

2. The lipidoid composition of claim 1, wherein each lipidoid independently comprises an amine head and a hydrophobic tail.

3. The lipidoid composition of claim 2, wherein each amine head independently comprises an alkylamine, an arylamine, a heteroarylamine, or a heterocyclylamine.

4. The lipidoid composition of claim 1 or 2, wherein each amine head independently comprises 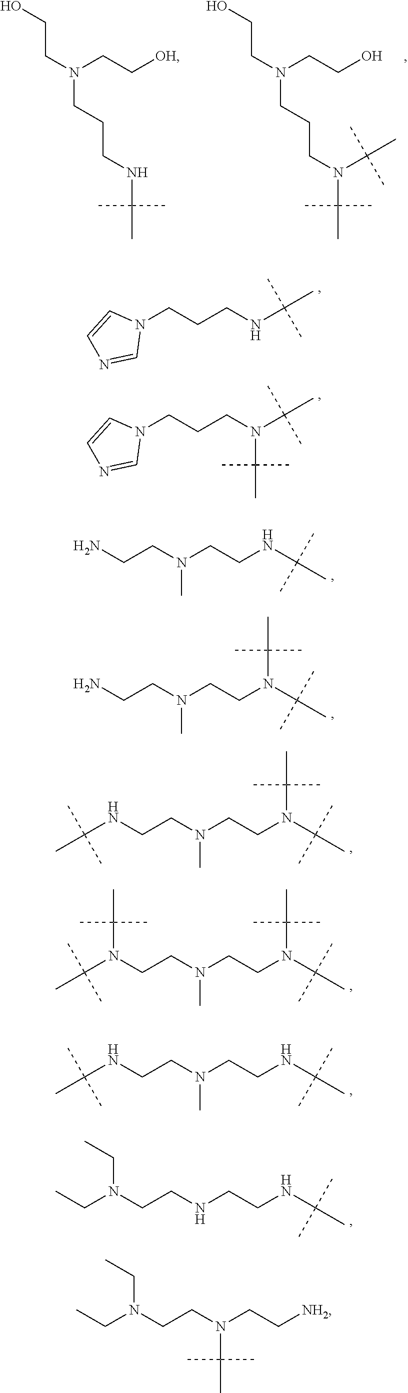 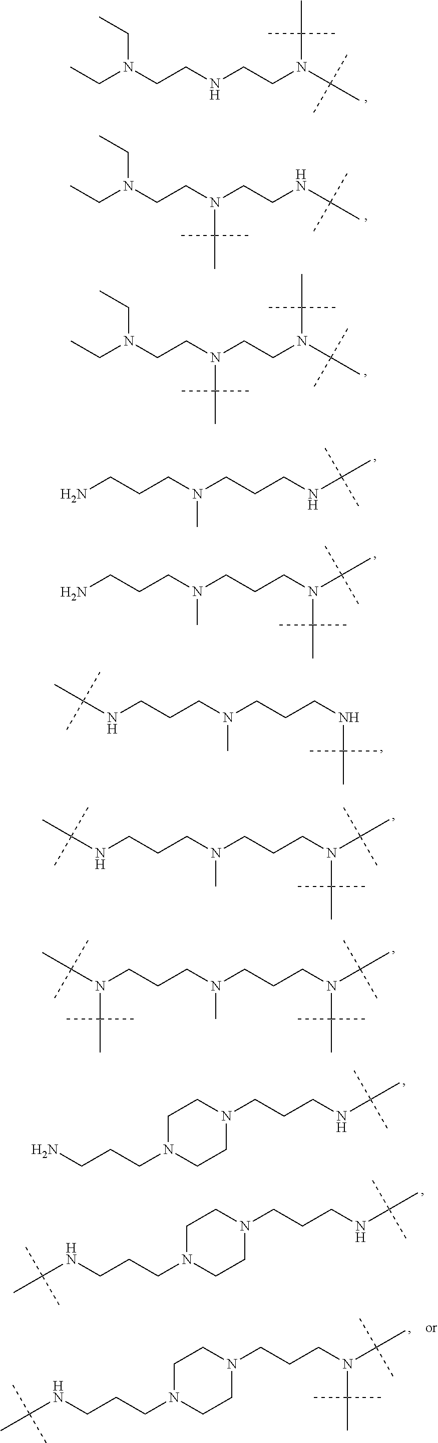 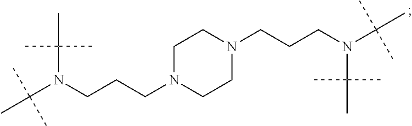
or a pharmaceutically acceptable salt thereof, and
each dashed line represents a connection (e.g., a bond) to the hydrophobic tail.

5. The lipidoid composition of any one of claims 2-4, wherein each amine head independently comprises 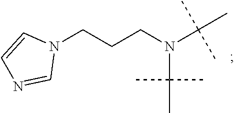
or a pharmaceutically acceptable salt thereof, and
each dashed line represents a connection (e.g., a bond) to the hydrophobic tail.

6. The lipidoid composition of any one of claims 2-4, wherein each amine head independently comprises 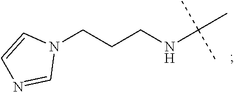
or a pharmaceutically acceptable salt thereof, and
the dashed line represents a connection (e.g., a bond) to the hydrophobic tail.

7. The lipidoid composition of claim 2 or 3, wherein each amine head independently consists essentially of 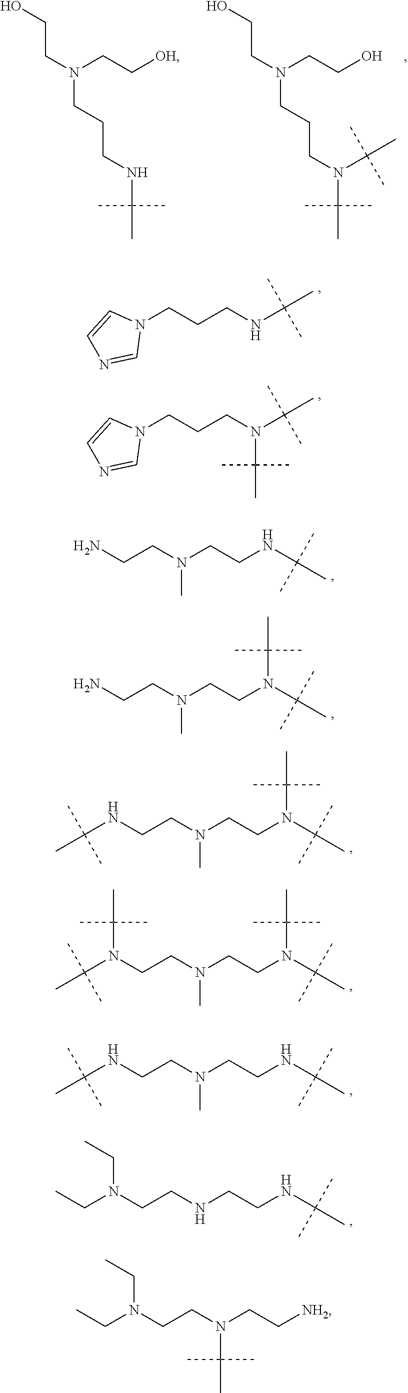 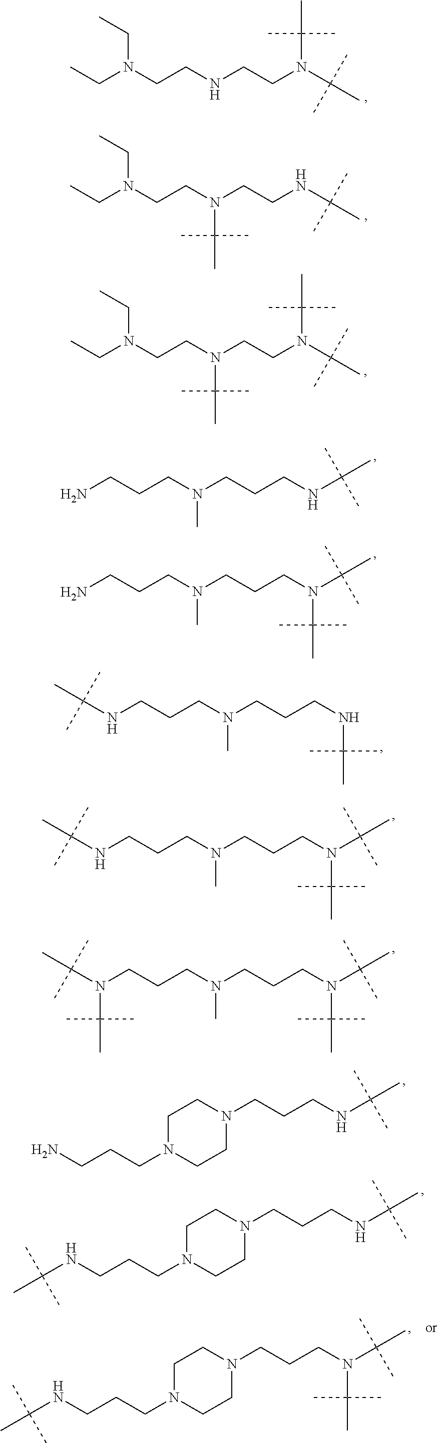 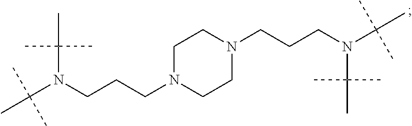
or a pharmaceutically acceptable salt thereof, and
each dashed line represents a connection (e.g., a bond) to the hydrophobic tail.

8. The lipidoid composition of claim 2 or 3, wherein each amine head independently consists essentially of 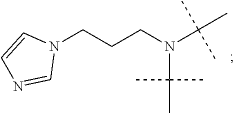
or a pharmaceutically acceptable salt thereof;
and
each dashed line represents a connection to the hydrophobic tail.

9. The lipidoid composition of claim 2 or 3, wherein each amine head independently consists essentially of 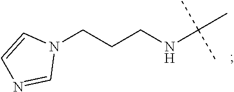
or a pharmaceutically acceptable salt thereof;
and
the dashed line represents a connection (e.g., a bond) to the hydrophobic tail.

10. The lipidoid composition of any one of claims 2-9, wherein each hydrophobic tail independently comprises a C₁-C₃₀ alkyl chain, a C₁-C₃₀ alkylacyl chain, a C₁-C₃₀ alkylester chain, or a C₁-C₃₀ alkylamide chain.

11. The lipidoid composition of any one of claims 2-9, wherein each hydrophobic tail independently comprises a C₁-C₃₀ alkyl chain, a C₁-C₃₀ alkylester chain, or a C₁-C₃₀ alkylamide chain.

12. The lipidoid composition of claim 10 or 11, wherein at least one carbon atom of the C₁-C₃₀ alkyl is replaced with a heteroatom.

13. The lipidoid composition of any one of claims 10-12, wherein 1, 2, 3, 4, 5, 6, 7, or 8 carbon atom(s) of the C₁-C₃₀ alkyl is replaced with a heteroatom.

14. The lipidoid composition of any one of claims 10-12, wherein 1 carbon atom of the C₁-C₃₀ alkyl is replaced with a heteroatom.

15. The lipidoid composition of any one of claims 10-12, wherein 2 carbon atoms of the C₁-C₃₀ alkyl is replaced with a heteroatom.

16. The lipidoid composition of any one of claims 13-15, wherein each heteroatom is independently selected from the group consisting of N (e.g., NH), 0, S, and Se.

17. The lipidoid composition of any one of claims 13-15, wherein each heteroatom is independently selected from the group consisting of O, S, and Se.

18. The lipidoid composition of any one of claims 13-15, wherein each heteroatom is independently selected from the group consisting of O, S, and Se.

19. The lipidoid composition of any one of claims 13-15, wherein each heteroatom is O.

20. The lipidoid composition of any one of claims 13-15, wherein each heteroatom is S.

21. The lipidoid composition of any one of claims 13-15, wherein each heteroatom is Se.

22. The lipidoid composition of any one of claims 2-21, wherein each hydrophobic tail is independently substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

23. The lipidoid composition of any one of claims 2-22, wherein each hydrophobic tail is independently substituted with halo (e.g., fluoro).

24. The lipidoid composition of any one of claims 2-9, wherein each hydrophobic tail comprises
the formula of 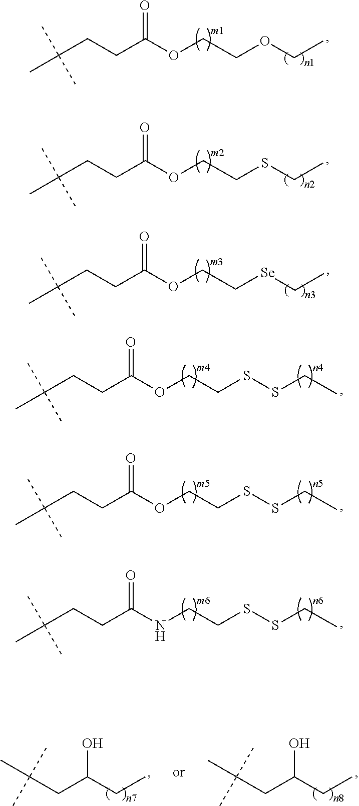
wherein the dashed line represents a connection (e.g., a bond) to the amine head, and wherein:
m1 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m2 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m3 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m4 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m5 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m6 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n1 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n2 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n3 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n4 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n5 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n6 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n7 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15); and
n8 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15).

25. The lipidoid composition of any one of claims 2-9, wherein each hydrophobic tail comprises 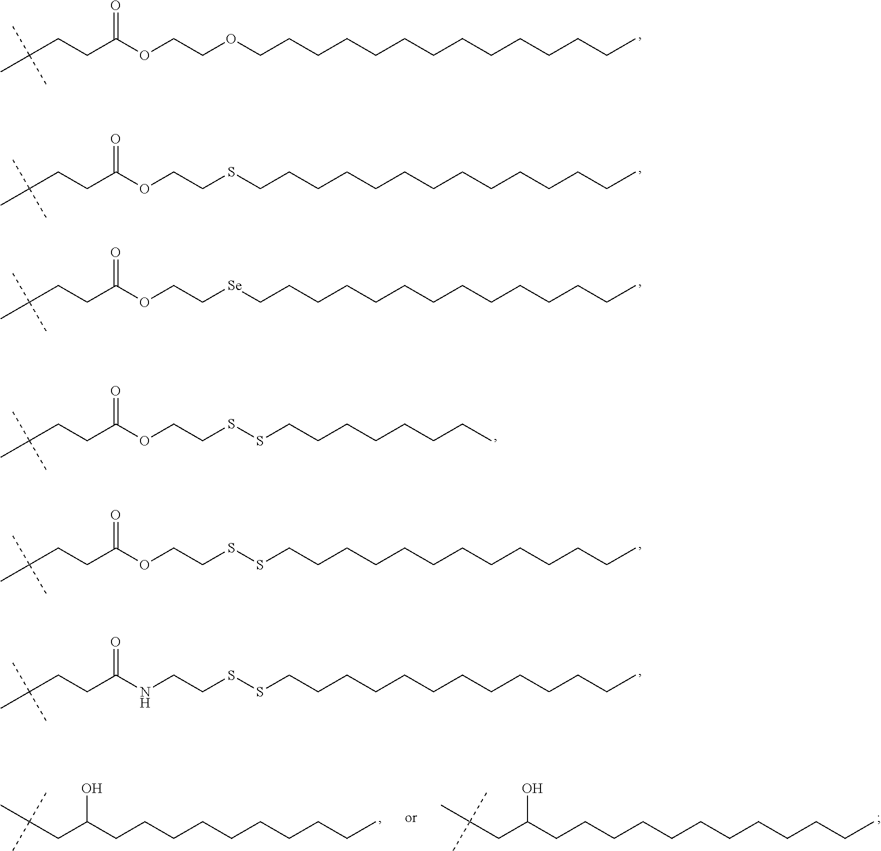
and
the dashed line represents a connection (e.g., a bond) to the amine head.

26. The lipidoid composition of any one of claims 2-9, wherein each hydrophobic tail comprises 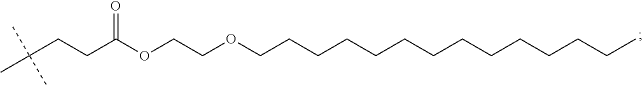
and
the dashed line represents a connection (e.g., a bond) to the amine head.

27. The lipidoid composition of claim 26, wherein the alkyl chain is substituted with halo (e.g., fluoro).

28. The lipidoid composition of any one of claims 2-9, wherein each hydrophobic tail comprises 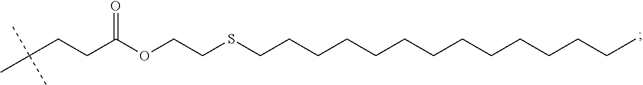
and
the dashed line represents a connection (e.g., a bond) to the amine head.

29. The lipidoid composition of claim 28, wherein the alkyl chain is substituted with halo (e.g., fluoro).

30. The lipidoid composition of any one of claims 2-9, wherein each hydrophobic tail consists essentially of the formula of 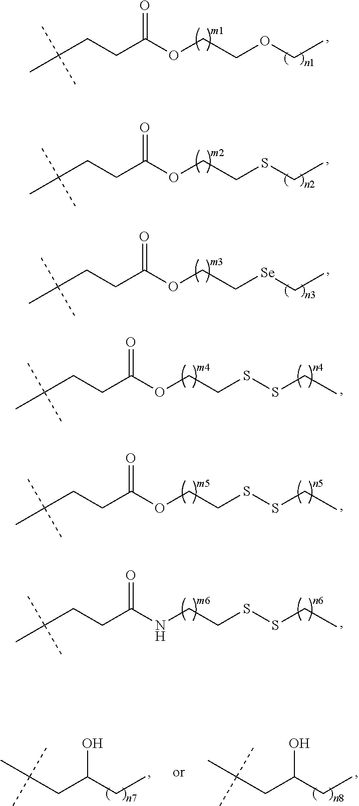
wherein the dashed line represents a connection (e.g., a bond) to the amine head, and wherein:
m1 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m2 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m3 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m4 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m5 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
m6 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n1 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n2 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n3 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n4 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n5 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n6 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15);
n7 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15); and
n8 is an integer from 4 to 30 (or any subrange thereof, e.g., from 4 to 25, from 4 to 25, from 6 to 25, from 4 to 20, from 6 to 20, from 4 to 18, from 6 to 18, from 4 to 15, or from 6 to 15).

31. The lipidoid composition of any one of claims 2-9, wherein each hydrophobic tail consists essentially of 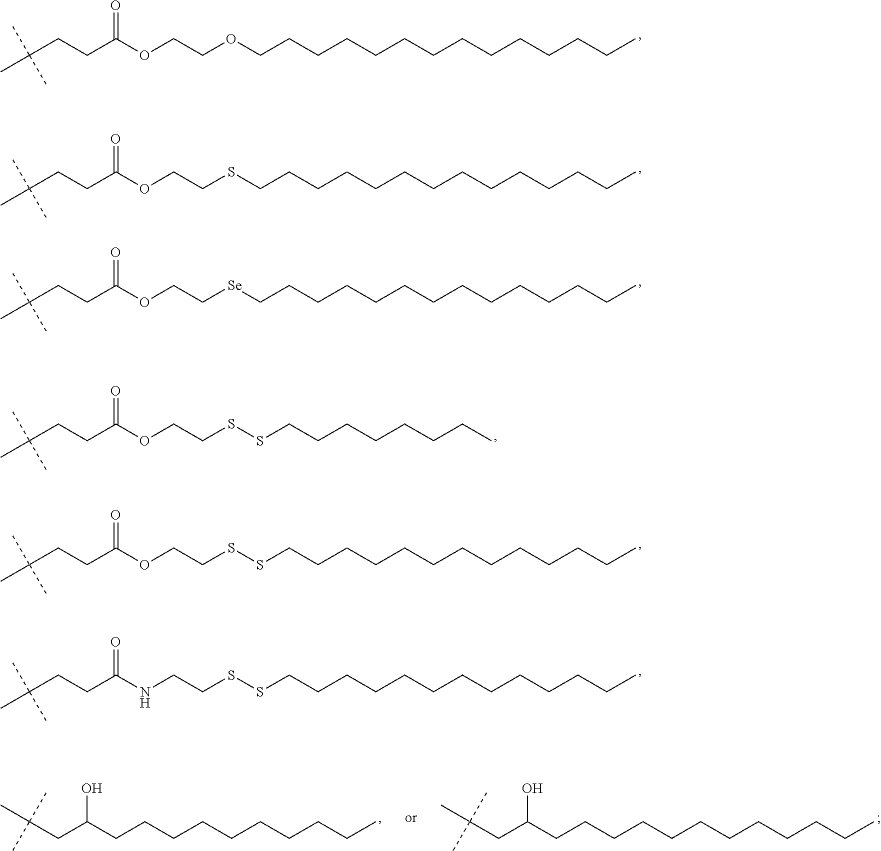
and
the dashed line represents a connection (e.g., a bond) to the amine head.

32. The lipidoid composition of any one of claims 2-9, wherein each hydrophobic tail consists essentially of 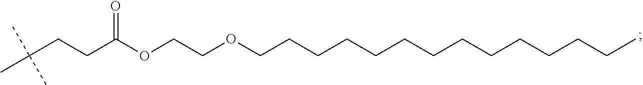
and
the dashed line represents a connection (e.g., a bond) to the amine head.

33. The lipidoid composition of claim 32, wherein the alkyl chain is substituted with halo (e.g., fluoro).

34. The lipidoid composition of any one of claims 2-9, wherein each hydrophobic tail consists essentially of 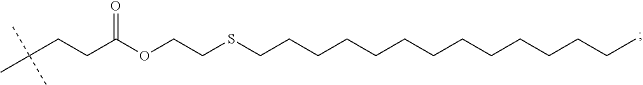
and
the dashed line represents a connection (e.g., a bond) to the amine head.

35. The lipidoid composition of claim 34, wherein the alkyl chain is substituted with halo (e.g., fluoro).

36. The lipidoid composition of any one of claims 1-35, wherein the plurality of lipidoids forms a bilayer.

37. The lipidoid composition of any one of claims 1-36, wherein the lipidoid composition comprises an adjuvant.

38. The lipidoid composition of claim 37, wherein the adjuvant is an immune modulator.

39. The lipidoid composition of claim 37, wherein the adjuvant is a stimulator of the immune system (e.g., a stimulator of the immune system of a mamma, such as a human).

40. The lipidoid composition of claim 39, wherein the stimulator of the immune system is an immune enhancer or immune inhibitor.

41. The lipidoid composition of claim 39, wherein the stimulator of the immune system stimulates innate immunity.

42. The lipidoid composition of claim 38, wherein the immune modulator is a stimulator of one or more immune-related genes, such as interferon genes (STING).

43. The lipidoid composition of claim 39, wherein the stimulator of the immune system is a stimulator of interferon genes (STING).

44. The lipidoid composition of claim 42 or 43, wherein the STING is a STING agonist.

45. The lipidoid composition of claim 38, wherein the immune modulator is an oligonucleotide, such as a (e.g., cyclic) dinucleotide.

46. The lipidoid composition of claim 45, wherein the STING agonist is a cyclic dinucleotide.

47. The lipidoid composition of claim 45, wherein the STING agonist is cyclic guanosine monophosphate-adenosine monophosphate (cGAMP).

48. The lipidoid composition of any one of claims 1-47, wherein the adjuvant is encapsulated within the lipidoid composition.

49. The lipidoid composition of any one of claims 36-48, wherein the plurality of lipidoids forms a bilayer and the adjuvant is encapsulated within the bilayer.

50. The lipidoid composition of any one of claims 1-39, wherein the lipidoid composition comprises an antigen.

51. The lipidoid composition of claim 50, wherein the antigen is a vaccine.

52. The lipidoid composition of claim 50 or 51, wherein the antigen is a protein.

53. The lipidoid composition of claim 50 or 51, wherein the antigen is an attenuated virus.

54. The lipidoid composition of any one of claims 50-53, wherein the antigen is encapsulated within the lipidoid composition.

55. The lipidoid composition of any one of claims 50-53, wherein the plurality of lipidoids forms a bilayer and the antigen is encapsulated within the bilayer.

56. The lipidoid composition of any one of claims 1-39, wherein the lipidoid composition comprises a nucleic acid.

57. The lipidoid composition of claim 56, wherein the nucleic acid is a DNA or a RNA.

58. The lipidoid composition of claim 56, wherein the nucleic acid is an RNA.

59. The lipidoid composition of claim 57 or 58, wherein the RNA is an mRNA.

60. The lipidoid composition of claim 59, wherein, when the mRNA contacts a cell, the mRNA induces the synthesis of a protein belonging to a cancer cell.

61. The lipidoid composition of claim 60, wherein the cancer cell is a bladder cancer cell, breast cancer cell, brain cancer cell, bone cancer cell, cervical cancer cell, colorectal cancer cell, head cancer cell, neck cancer cell, kidney cancer cell, liver cancer cell, lung cancer cell, lymphoma cell, mesothelioma cell, myeloma cell, prostate cancer cell, skin cancer cell, thyroid cancer cell, ovarian cancer cell, or uterine cancer cell.

62. The lipidoid composition of claim 58, wherein, when the mRNA contacts a cell, the mRNA induces the synthesis of a protein belonging to a virus.

63. The lipidoid composition of claim 62, wherein the virus is hepatitis C, norovirus, junin, dengue virus, coronavirus, human immunodeficiency virus, herpes simplex, avian flu, chickenpox, cold sores, common cold, glandular fever, influenza, measles, mumps, pharyngitis, pneumonia, rubella, severe acute respiratory syndrome, and lower or upper respiratory tract infection (e.g., respiratory syncytial virus).

64. The lipidoid composition of claim 62, wherein the virus is an influenza virus.

65. The lipidoid composition of claim 62, wherein the virus is a human immunodeficiency virus.

66. The lipidoid composition of claim 62, wherein the virus is a coronavirus.

67. The lipidoid composition of claim 62, wherein the coronavirus is SARS-CoV-2.

68. The lipidoid composition of claim 67, wherein the SARS-CoV-2 is the alpha, beta, gamma, delta, or omicron strain of SARS-CoV-2.

69. The lipidoid composition of claim 67 or 68, wherein, when the mRNA contacts a cell, the mRNA induces the synthesis of the spike protein of the SARS-CoV-2.

70. The lipidoid composition of claim 59, wherein the mRNA has at least 60%, 65%, 70%, 75%, 80%, 85%, 90%, 95%, or 99% sequence identity to a sequence depicted in FIG. 12.

71. The lipidoid composition of claim 59, wherein the mRNA has at least 75%, 80%, 85%, 90%, 95%, or 99% sequence identity to a sequence depicted in FIG. 12.

72. The lipidoid composition of claim 59, wherein the mRNA has at least 90%, 95%, or 99% sequence identity to a sequence depicted in FIG. 12.

73. The lipidoid composition of claim 59, wherein the mRNA has at least 95%, or 99% sequence identity to a sequence depicted in FIG. 12.

74. The lipidoid composition of claim 59, wherein the mRNA has a sequence according to a sequence depicted in FIG. 12.

75. The lipidoid composition of any one of claims 56-74, wherein the nucleic acid is encapsulated within the lipidoid composition.

76. The lipidoid composition of any one of claims 56-74, wherein the plurality of lipidoids forms a bilayer and the nucleic is encapsulated within the bilayer.

77. The lipidoid composition of any one of claims 1-61, wherein the lipidoid composition further comprises a chemotherapeutic agent.

78. The lipidoid composition of claim 77, wherein the chemotherapeutic agent is cytotoxic.

79. The lipidoid composition of claim 77 or 78, wherein the chemotherapeutic agent is an alkylating agent, an antimetabolite, an anti-microtubule agent, anti-tumor antibiotic, or a topoisomerase inhibitor, a mitotic inhibitor, or a corticosteroid.

80. The lipidoid composition of any one of claims 77-79, wherein the chemotherapeutic agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-Fu), 6-mercaptopurine (6-MP), capecitabine, cladribine, clofarabine, cytarabine (Ara-C), decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, doxorubicin liposomal, epirubicin, idarubicin, valrubicin, bleomycin, dactinomycin, mitomycin-c, mitoxantrone, irinotecan, irinotecan liposomal, topotecan, etoposide, mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, or vorinostat.

81. The lipidoid composition of any one of claims 77-80, wherein the chemotherapeutic agent is doxorubicin.

82. The lipidoid composition of any one of claims 77-81, wherein the chemotherapeutic agent is encapsulated within the lipidoid composition.

83. The lipidoid composition of any one of claims 1-82, wherein the lipidoid compositions are capable of internalizing an antigen (e.g., an antigen from a cancer cell).

84. A pharmaceutical composition comprising a lipidoid composition of any one of claims 1-83 and a pharmaceutically acceptable excipient.

85. The pharmaceutical composition of claim 84, wherein the compositions is formulated for delivery to an immune cell of subject (e.g., a human subject).

86. The pharmaceutical composition of claim 84 or 85, wherein the composition further comprises an antigen.

87. The pharmaceutical composition of any one of claims 84-86, wherein the composition further comprises an immune modulator.

88. A method of treating or preventing a disease or disorder in a subject in need thereof comprising administering a therapeutically effective amount of the lipidoid composition of any one of claims 1-83 to the subject.

89. A method of treating or preventing cancer in a subject in need thereof comprising administering a therapeutically effective amount of the lipidoid composition of any one of claims 1-83 to the subject.

90. A method of treating or preventing cancer in a subject in need thereof, comprising the steps of administering a therapeutically effective amount of a chemotherapeutic agent to the subject; and administering a therapeutically effective amount of the lipidoid composition of claim 1-83 to the subject.

91. The method of claim 89 or 90, wherein the method treats the cancer.

92. The method of claim 89 or 90, wherein the method prevents the cancer.

93. The method of any one of claims 90-92, wherein the chemotherapeutic agent is cytotoxic.

94. The method of any one of claims 90-93, wherein the chemotherapeutic agent is an alkylating agent, an antimetabolite, an anti-microtubule agent, anti-tumor antibiotic, or a topoisomerase inhibitor, a mitotic inhibitor, or a corticosteroid.

95. The method of claim 94, wherein the chemotherapeutic agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-Fu), 6-mercaptopurine (6-MP), capecitabine, cladribine, clofarabine, cytarabine (Ara-C), decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, doxorubicin liposomal, epirubicin, idarubicin, valrubicin, bleomycin, dactinomycin, mitomycin-c, mitoxantrone, irinotecan, irinotecan liposomal, topotecan, etoposide, mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, or vorinostat.

96. The method of claim 94, wherein the chemotherapeutic agent is doxorubicin.

97. The method of any one of claims 90-96, wherein the lipidoid composition is administered intratumorally.

98. The method of any one of claims 90-97, wherein the lipidoid composition is administered about 6-48 hours after the chemotherapeutic agent.

99. The method of any one of claims 90-97, wherein the lipidoid composition is administered about 6-24 hours after the chemotherapeutic agent.

100. The method of any one of claims 90-97, wherein the lipidoid composition is administered about 24 hours after the chemotherapeutic agent.

101. The method of any one of claims 89-100, wherein the cancer is a solid tumor.

102. The method of any one of claims 89-100, wherein the cancer is bladder cancer, breast cancer, brain cancer, bone cancer, cervical cancer, colorectal cancer, head cancer, neck cancer, kidney cancer, liver cancer, lung cancer, lymphoma, mesothelioma, myeloma, prostate cancer, skin cancer, thyroid cancer, ovarian cancer, or uterine cancer.

103. The method of any one of claims 89-102, wherein the method elicits an anti-cancer immune response in the subject.

104. A method of treating or preventing a viral infection in a subject in need thereof comprising administering a therapeutically effective amount of the lipidoid composition of any one of claims 1-83 to the subject.

105. The method of claim 104, wherein the method treats the viral infection.

106. The method of claim 104, wherein the method prevents the viral infection.

107. The lipidoid composition of claim 104-106, wherein the viral infection is hepatitis C, norovirus, junin, dengue virus, coronavirus, human immunodeficiency virus, herpes simplex, avian flu, chickenpox, cold sores, common cold, glandular fever, influenza, measles, mumps, pharyngitis, pneumonia, rubella, severe acute respiratory syndrome, and lower or upper respiratory tract infection (e.g., respiratory syncytial virus).

108. The lipidoid composition of claim 107, wherein the viral infection is an influenza virus.

109. The lipidoid composition of claim 107, wherein the viral infection is a human immunodeficiency virus.

110. The lipidoid composition of claim 107, wherein the viral infection is a coronavirus.

111. The lipidoid composition of claim 110, wherein the coronavirus is SARS-CoV-2.

112. The lipidoid composition of claim 111, wherein the SARS-CoV-2 is the alpha, beta, gamma, delta, or omicron strain of SARS-CoV-2.

113. The method of any one of claims 104-112, wherein the method elicits an immune response in the subject.

114. The method of any one of claims 104-112, wherein the method elicits an antiviral immune response in the subject.

115. The method of any one of claims 104-114, wherein the method vaccinates the subject against the viral infection.

116. A kit comprising chemotherapeutic agent and a lipidoid composition of any one of claims 1-83

117. A kit comprising chemotherapeutic agent and a pharmaceutical composition of claim any one of claims 84-87.

118. The kit of claim 116 or 117, wherein the chemotherapeutic agent is cytotoxic.

119. The kit of any one of claims 116-118, wherein the chemotherapeutic agent is an alkylating agent, an antimetabolite, an anti-microtubule agent, anti-tumor antibiotic, or a topoisomerase inhibitor, a mitotic inhibitor, or a corticosteroid.

120. The kit of any one of claims 116-118, wherein the chemotherapeutic agent is the chemotherapeutic agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, carmustine, lomustine, streptozocin, azacitidine, 5-fluorouracil (5-Fu), 6-mercaptopurine (6-MP), capecitabine, cladribine, clofarabine, cytarabine (Ara-C), decitabine, floxuridine, fludarabine, gemcitabine, hydroxyurea, methotrexate, nelarabine, pemetrexed, pentostatin, pralatrexate, thioguanine, trifluridine, tipiracil, daunorubicin, doxorubicin, doxorubicin liposomal, epirubicin, idarubicin, valrubicin, bleomycin, dactinomycin, mitomycin-c, mitoxantrone, irinotecan, irinotecan liposomal, topotecan, etoposide, mitoxantrone, teniposide, cabazitaxel, docetaxel, nab-paclitaxel, paclitaxel, vinblastine, vincristine, vincristine liposomal, vinorelbine, prednisone, methylprednisolone, dexamethasone, retinoic acid, arsenic trioxide, asparaginase, eribulin, hydroxyurea, ixabepilone, mitotane, omacetaxine, pegaspargase, procarbazine, romidepsin, or vorinostat.

121. The kit of claim 120, wherein the chemotherapeutic agent is doxorubicin.
